(19) United States
(12) Patent Application Publication
(10) Pub. No.: US 2025/0195901 A1
(43) Pub. Date: Jun. 19, 2025

(54) BASELINING THERAPY ENERGY FOR WEARABLE CARDIAC TREATMENT DEVICES

(71) Applicant: ZOLL Medical Corporation, Chelmsford, MA (US)

(72) Inventors: Bahar Davoudi, Pittsburgh, PA (US); Gary A. Freeman, Waltham, MA (US); Matthew L. Sundermann, Pittsburgh, PA (US); Steven J. Szymkiewicz, Venetia, PA (US); Kent J. Volosin, Big Sky, MT (US)

(73) Assignee: ZOLL Medical Corporation, Chelmsford, MA (US)

(21) Appl. No.: 18/845,164

(22) PCT Filed: Mar. 8, 2023
(86) PCT No.: PCT/US2023/063946

§ 371 Date: Sep. 9, 2024

Related U.S. Application Data
(60) Provisional application No. 63/269,142, filed on Mar. 10, 2022.

(51) Int. Cl. A61N 1/39; A61B 5/00; A61B 5/256; A61B 5/355; A61B 5/361; A61B 5/364; A61N 1/02; A61N 1/04; A61N 1/36; G16H 20/30

(52) U.S. Cl. CPC A61N 1/3904; A61B 5/256; A61B 5/355; A61B 5/361; A61B 5/364; A61B 5/4836; A61B 5/6804; A61N 1/025; A61N 1/046; A61N 1/0484; A61N 1/36031; A61N 1/36034; A61N 1/3925; G16H 20/30; A61B 2560/0468

(57) ABSTRACT

A wearable cardiac treatment system configured to treat arrhythmias occurring in a patient is provided. The system includes a garment, a plurality of ECG electrodes, and a plurality of therapy electrodes, and a memory. The system also a cardiac controller including one or more processors configured to determine one or more timing parameters and/or morphology parameters of ECG signals of the patient and apply cardiac rhythm disruptive shock(s) at predetermined one or more times based on the one or more timing parameters and/or morphology parameters. The cardiac rhythm disruptive shock(s) are delivered at same and/or decreasing energy levels until a cardiac rhythm change is induced in the patient. The one or more processors are also configured to detect the cardiac rhythm change, record an energy level of a shock that induced the cardiac rhythm change, and adjust a defibrillation energy level for future defibrillation shock(s) based on the energy level.

### CROSS-REFERENCE TO RELATED APPLICATIONS

This application claims priority to U.S. Provisional Patent Application Ser. No. 63/269,142, filed on Mar. 10, 2022, titled “BASELINING THERAPY ENERGY FOR WEARABLE CARDIAC TREATMENT DEVICES,” the entirety of which is hereby incorporated by reference.

### BACKGROUND

The present disclosure relates to a wearable cardiac treatment system configured to treat cardiac arrhythmias occurring in ambulatory and/or in-hospital patients.

Heart failure, if left untreated, can lead to certain life-threatening arrhythmias. Both atrial and ventricular arrhythmias are common in patients with heart failure. One of the deadliest cardiac arrhythmias is ventricular fibrillation, which occurs when normal, regular electrical impulses are replaced by irregular and rapid impulses, causing the heart muscle to stop normal contractions. Because the victim has no perceptible warning of the impending fibrillation, death often occurs before the necessary medical assistance can arrive. Other cardiac arrhythmias can include excessively slow heart rates known as bradycardia or excessively fast heart rates known as tachycardia. Cardiac arrest can occur when a patient experiences various arrhythmias of the heart, such as ventricular fibrillation, ventricular tachycardia, pulseless electrical activity (PEA), and asystole (heart stops all electrical activity), result in the heart providing insufficient levels of blood flow to the brain and other vital organs for the support of life. It is generally useful to monitor heart failure patients to assess heart failure symptoms early and provide interventional therapies as soon as possible.

Patients may be prescribed to wear cardiac treatment devices for extended periods of time. Cardiac treatment devices may provide defibrillation shocks to the patients if an abnormal cardiac rhythm is detected. The energy level of the defibrillation shocks is set to ensure that patients are effectively treated if they experience an abnormal cardiac rhythm.

### SUMMARY

In one or more examples, a wearable cardiac treatment system configured to treat arrhythmias occurring in an ambulatory patient is provided. The system includes a garment configured to be worn about a torso of the ambulatory patient, a plurality of ECG electrodes configured to be disposed on the garment and further configured to sense ECG signals indicative of cardiac activity in the patient, and a plurality of therapy electrodes configured to be disposed on the garment. The system also includes a memory configured to store baseline therapy energy information and a cardiac controller including one or more processors in communication with the memory, the plurality of ECG electrodes and the plurality of therapy electrodes. The one or more processors are configured to determine one or more timing parameters and/or one or more morphology parameters of the ECG signals of the patient and apply, via the one or more therapy electrodes, at least one or a series of cardiac rhythm disruptive shocks at predetermined one or more times based on the one or more timing parameters and/or one or more morphology parameters of the ECG signals of the patient. The at least one or series of the cardiac rhythm disruptive shocks are delivered at same and/or decreasing energy levels until a cardiac rhythm change is induced in the patient. The one or more processors are also configured to detect the cardiac rhythm change in the patient; record, in the memory, an energy level of a cardiac rhythm disruptive shock that induced the cardiac rhythm change as the baseline therapy energy information; and adjust a defibrillation energy level for one or more future defibrillation shocks to be delivered by the wearable cardiac treatment system based on the energy level of the cardiac rhythm disruptive shock that induced the cardiac rhythm change.

Implementations of the wearable cardiac treatment system can include one or more of the following features. The cardiac controller further includes the memory configured to store the baseline therapy energy information. The wearable cardiac treatment system further includes a remote server including the memory configured to store the baseline therapy energy information. The cardiac rhythm change in the patient includes ventricular fibrillation. The cardiac rhythm change in the patient includes a premature ventricular contraction. The one or more timing parameters and/or one or more morphology parameters of the ECG signals include one or more timings and/or one or more morphologies corresponding to one or more T-waves in the patient. The at least one or the series of cardiac rhythm disruptive shocks include at least one or a series of pacing pulses. The one or more processors are configured to apply the at least one or the series of pacing pulses asynchronously with a normal cardiac rhythm of the patient. The one or more processors are further configured to deliver, via the one or more therapy electrodes, a plurality of pacing pulses configured to pace a heart of the patient according to a predetermined cardiac rhythm. The one or more processors are configured to determine the predetermined one or more times based on the one or more timing parameters and/or one or more morphology parameters of the ECG signals and further based on the predetermined cardiac rhythm.

The one or more processors are configured to apply, via the one or more therapy electrodes, the at least one or the series of cardiac rhythm disruptive shocks by applying a first cardiac rhythm disruptive shock at a first energy level and detecting that no cardiac rhythm change has occurred in the patient. The one or more processors are further configured to apply, via the one or more therapy electrodes, the at least one or the series of cardiac rhythm disruptive shocks by applying a second cardiac rhythm disruptive shock at a second energy level lower than the first energy level. The first energy level includes an energy level between around 20 to 90 Joules. The first energy level includes an energy level between around 70 to 90 Joules. The first energy level includes an energy level between around 20 to 90 Joules. The second energy level includes an energy level between around 30 to 50 Joules.

The one or more processors are configured to apply, via the one or more therapy electrodes, the at least one or the series of cardiac rhythm disruptive shocks by applying a first cardiac rhythm disruptive shock at a first energy level, the first cardiac rhythm disruptive shock inducing the cardiac rhythm change in the patient. The one or more processors are further configured to apply, via the one or more therapy electrodes, a second cardiac rhythm disruptive shock at a second energy level higher than the first energy level. The first energy level includes an energy level between around 20 to 90 Joules. The first energy level includes an energy level between around 70 to 90 Joules. The second energy level includes an energy level between around 20 to 90 Joules. The second energy level includes an energy level between around 30 to around 50 Joules.

The one or more processors are further configured to apply, via the one or more therapy electrodes, a cardiac rhythm restoring shock to the patient to restore a normal cardiac rhythm on detecting the cardiac rhythm change in the patient. The cardiac rhythm restoring shock includes a defibrillation shock. The one or more processors are configured to apply, via the one or more therapy electrodes, the cardiac rhythm restoring shock to restore the normal cardiac rhythm by applying a first cardiac rhythm restoring shock at a first restoring shock energy level, detecting that the patient has not been restored to the normal cardiac rhythm, and applying a second cardiac rhythm restoring shock at a second restoring shock energy level higher than the first restoring shock energy level. The one or more processors are further configured to record, in the memory, a restoring shock energy level of the cardiac rhythm restoring shock that restored the patient to the normal cardiac rhythm. The one or more processors are configured to further adjust the defibrillation energy level for one or more future defibrillation shocks to be delivered by the wearable cardiac treatment system based on the restoring shock energy level of the cardiac rhythm restoring shock that restored the patient to the normal cardiac rhythm. The first restoring shock energy level includes an energy level between around 20 to around 90 Joules. The second restoring shock energy level includes an energy level between around 20 to around 90 Joules. The one or more processors are configured to apply, via the one or more therapy electrodes, the cardiac rhythm restoring shock after a predetermined delay. The predetermined delay includes a time between around 10 ms to around 40 ms. The predetermined delay is user-configurable.

The one or more processors are further configured to repeat applying the at least one or the series of cardiac rhythm disruptive shocks, detecting the cardiac rhythm change in the patient, and recording the energy level of the cardiac rhythm disruptive shock that induced the cardiac rhythm change. The one or more processors are further configured to construct a dose-response curve for the patient based on the recorded energy levels of the cardiac rhythm disruptive shocks that induced the cardiac rhythm changes. The one or more processors are further configured to determine a predetermined percentile of the dose-response curve. The predetermined percentile includes a 50th percentile. The one or more processors are configured to adjust the defibrillation energy level for the one or more future defibrillation shocks to be delivered by the wearable cardiac treatment system using an energy level corresponding to the predetermined percentile of the dose-response curve. The one or more processors are configured to adjust the energy level for the one or more future defibrillation shocks by performing a statistical analysis on the energy levels of the cardiac rhythm disruptive shocks that induced the cardiac rhythm changes and adjusting the defibrillation energy level for the one or more future defibrillation shocks based on the statistical analysis. Performing the statistical analysis on the energy levels of the cardiac disruptive shocks that induced the cardiac rhythm changes includes finding at least one of an average, a median, or a highest energy level of the energy levels of the cardiac disruptive shocks that induced the cardiac rhythm changes. Performing the statistical analysis on the cardiac disruptive shocks that induced the cardiac rhythm changes includes eliminating any outlier cardiac disruptive shocks that induced the cardiac rhythm changes. Finding the at least one of the average, the median, or the highest energy level of the cardiac disruptive shocks that induced the cardiac rhythm changes includes finding an average, a median, or a highest energy level of the energy levels of the cardiac disruptive shocks that induced the cardiac rhythm changes remaining after eliminating any outlier cardiac disruptive shocks that induced the cardiac rhythm changes.

In one or more examples, a wearable cardiac treatment system configured to treat arrhythmias occurring in an ambulatory patient is provided. The system includes a garment configured to be worn about a torso of the ambulatory patient, a plurality of ECG electrodes configured to be disposed on the garment and configured to sense ECG signals indicative of cardiac activity in the patient, and a plurality of therapy electrodes configured to be disposed on the garment. The system also includes a memory configured to store baseline therapy energy information and a cardiac controller including one or more processors in communication with the memory, the plurality of ECG electrodes, and the plurality of therapy electrodes. The one or more processors are configured to determine one or more timing parameters and/or one or more morphology parameters of T-waves in the patient based on the sensed ECG signals and apply, via the one or more therapy electrodes, at least one or a series of fibrillation shocks at predetermined one or more times based on the one or more timing parameters and/or one or more morphology parameters of the T-waves in the patient. The at least one or the series of fibrillation shocks are delivered at same and/or decreasing energy levels until the patient goes into a ventricular fibrillation state. The one or more processors are also configured to detect that the patient is in the ventricular fibrillation state; apply, via the one or more therapy electrodes, a defibrillation shock to the patient to treat the ventricular fibrillation state on detecting that the patient is in the ventricular fibrillation state; record, in the memory, an energy level of a fibrillation shock that induced the ventricular fibrillation state as the baseline therapy energy information; and adjust a defibrillation energy level for one or more future defibrillation shocks to be delivered by the wearable cardiac treatment system based on the energy level of the fibrillation shock that induced the ventricular fibrillation state.

Implementations of the wearable cardiac treatment system can include one or more of the applicable features discussed with respect to the wearable cardiac treatment system above and/or one or more of the following features. The cardiac controller further includes the memory configured to store the baseline therapy energy information. A remote server including the memory configured to store the baseline therapy energy information. The one or more processors are further configured to deliver, via the one or more therapy electrodes, a plurality of pacing pulses configured to pace a heart of the patient according to a predetermined cardiac rhythm. The one or more processors are configured to determine the predetermined one or more times based on the one or more timing parameters and/or one or more morphology parameters of the T-waves in the patient and further based on the predetermined cardiac rhythm.

The one or more processors are further configured to repeat applying the at least one or the series of fibrillation shocks, detecting the ventricular fibrillation state, and recording the energy level of the fibrillation shock that induced the ventricular fibrillation state. The one or more processors are configured to adjust the energy level for the one or more future defibrillation shocks by performing a statistical analysis on the fibrillation shocks that induced the ventricular fibrillation state and adjusting the defibrillation energy level for the one or more future defibrillation shocks based on the statistical analysis.

A method for treating arrhythmias occurring in an ambulatory patient using a wearable cardiac treatment system is provided. The method includes sensing ECG signals indicative of cardiac activity in the patient using a plurality of ECG electrodes of the wearable cardiac treatment system, determining one or more timing parameters and/or one or more morphology parameters of the ECG signals of the patient, and applying at least one or a series of cardiac rhythm disruptive shocks at predetermined one or more times using a plurality of therapy electrodes of the wearable cardiac treatment system, based on the one or more timing parameters and/or one or more morphology parameters of the ECG signals of the patient, at same and/or decreasing energy levels until a cardiac rhythm change is induced in the patient. The method also includes detecting the cardiac rhythm change in the patient, recording an energy level of a cardiac rhythm disruptive shock that induced the cardiac rhythm change as baseline therapy energy information in a memory of the wearable cardiac treatment system, and adjusting a defibrillation energy level for one or more future defibrillation shocks to be delivered by the wearable cardiac treatment system based on the energy level of the cardiac rhythm disruptive shock that induced the cardiac rhythm change.

Implementations of the method for treating arrhythmias occurring in an ambulatory patient using a cardiac treatment system can include one or more of the applicable features discussed with respect to the wearable cardiac treatment systems above and/or one or more of the following features. The plurality of ECG electrodes are configured to be disposed on a garment worn about a torso of the ambulatory patient. The plurality of therapy electrodes are configured to be disposed on the garment. The wearable cardiac treatment system includes a cardiac controller including the memory. The wearable cardiac treatment system includes a remote server including the memory. The cardiac rhythm change in the patient includes ventricular fibrillation. The cardiac rhythm change in the patient includes a premature ventricular contraction. The one or more timing parameters and/or one or more morphology parameters of the ECG signals include one or more timings and/or one or more morphologies corresponding to one or more T-waves in the patient. The at least one or the series of cardiac rhythm disruptive shocks include at least one or a series of pacing pulses. Applying the at least one or the series of cardiac rhythm disruptive shocks at the predetermined one or more times includes applying the at least one or the series of pacing pulses asynchronously with a normal cardiac rhythm of the patient. The method further includes delivering a plurality of pacing pulses configured to pace a heart of the patient according to a predetermined cardiac rhythm. The method further includes determining the predetermined one or more times based on the one or more timing parameters and/or one or more morphology parameters of the ECG signals and further based on the predetermined cardiac rhythm.

Applying the at least one or the series of cardiac rhythm disruptive shocks at the predetermined one or more times using the plurality of therapy electrodes of the wearable cardiac treatment system includes applying a first cardiac rhythm disruptive shock at a first energy level and detecting that no cardiac rhythm change has occurred in the patient. Applying the at least one or the series of cardiac rhythm disruptive shocks at the predetermined one or more times using the plurality of therapy electrodes of the wearable cardiac treatment system includes applying a second cardiac rhythm disruptive shock at a second energy level lower than the first energy level. Applying the at least one or the series of cardiac rhythm disruptive shocks at the predetermined one or more times using the plurality of therapy electrodes of the wearable cardiac treatment system includes applying a first cardiac rhythm disruptive shock at a first energy level, the first cardiac rhythm disruptive shock inducing the cardiac rhythm change in the patient. The method further includes using the plurality of therapy electrodes, a second cardiac rhythm disruptive shock at a second energy level higher than the first energy level.

The method further includes applying, using the plurality of therapy electrodes, a cardiac rhythm restoring shock to the patient to restore a normal cardiac rhythm on detecting the cardiac rhythm change in the patient. The rhythm restoring shock includes a defibrillation shock. Applying, using the plurality of therapy electrodes, the cardiac rhythm restoring shock includes applying a first cardiac rhythm restoring shock at a first restoring shock energy level, detecting that the patient has not been restored to the normal cardiac rhythm, and applying a second cardiac rhythm restoring shock at a second restoring shock energy level higher than the first restoring shock energy level. The method further includes recording, in the memory, a restoring shock energy level of the cardiac rhythm restoring shock that restored the patient to the normal cardiac rhythm. Adjusting the defibrillation energy level for the one or more future defibrillation shocks to be delivered by the wearable cardiac treatment system includes adjusting the defibrillation energy level for the one or more future defibrillation shocks to be delivered by the wearable cardiac treatment system based on the energy level of the cardiac rhythm disruptive shock that induced the cardiac rhythm change and further based on the restoring shock energy level of the cardiac rhythm restoring shock that restored the patient to the normal cardiac rhythm.

The method further includes applying the at least one or the series of cardiac rhythm disruptive shocks, detecting the cardiac rhythm change in the patient, and recording the energy level of the cardiac rhythm disruptive shock that induced the cardiac rhythm change. The method further includes constructing a dose-response curve for the patient based on the recorded energy levels of the cardiac rhythm disruptive shocks that induced the cardiac rhythm changes. The method further includes determining a predetermined percentile of the dose-response curve. The predetermined percentile includes a 50th percentile. Adjusting the defibrillation energy level for the one or more future defibrillation shocks to be delivered by the wearable cardiac treatment system includes adjusting the defibrillation energy level for the one or more future defibrillation shocks to be delivered by the wearable cardiac treatment system using an energy level corresponding to the predetermined percentile of the dose-response curve. Adjusting the defibrillation energy level for the one or more future defibrillation shocks to be delivered by the wearable cardiac treatment system includes performing a statistical analysis on the energy levels of the cardiac rhythm disruptive shocks that induced the cardiac rhythm changes and adjusting the defibrillation energy level for the one or more future defibrillation shocks based on the statistical analysis.

In one or more examples, a non-transitory computer-readable medium storing sequences of instructions executable by at least one processor is provided. The sequences of instructions instruct the at least one processor to treat arrhythmias occurring in an ambulatory patient using a wearable cardiac treatment system. The sequences of instructions include instructions to sense ECG signals indicative of cardiac activity in the patient using a plurality of ECG electrodes of the wearable cardiac treatment system, determine one or more timing parameters and/or one or more morphology parameters of the ECG signals of the patient, and apply at least one or a series of cardiac rhythm disruptive shocks at predetermined one or more times using a plurality of therapy electrodes of the wearable cardiac treatment system, based on the one or more timing parameters and/or one or more morphology parameters of the ECG signals of the patient, at same and/or decreasing energy levels until a cardiac rhythm change is induced in the patient. The sequences of instructions further include instructions to detect the cardiac rhythm change in the patient, record an energy level of a cardiac rhythm disruptive shock that induced the cardiac rhythm change as baseline therapy energy information in a memory of the wearable cardiac treatment system, and adjust a defibrillation energy level for one or more future defibrillation shocks to be delivered by the wearable cardiac treatment system based on the energy level of the cardiac rhythm disruptive shock that induced the cardiac rhythm change.

Implementations of the non-transitory computer-readable medium storing sequences of instructions executable by the at least one processor one or more of the applicable features discussed with respect to the wearable cardiac treatment systems above and/or one or more of the following features. The wearable cardiac treatment system includes a cardiac controller including the memory. The wearable cardiac treatment system includes a remote server including the memory. The cardiac rhythm change in the patient includes ventricular fibrillation. The cardiac rhythm change in the patient includes a premature ventricular contraction. The one or more timing parameters and/or one or more morphology parameters of the ECG signals include one or more timings and/or one or more morphologies corresponding to one or more T-waves in the patient. The at least one or the series of cardiac rhythm disruptive shocks include at least one or a series of pacing pulses. The instructions to apply the at least one or the series of cardiac rhythm disruptive shocks at the predetermined one or more times further include instructions to apply the at least one or the series of pacing pulses asynchronously with a normal cardiac rhythm of the patient. The sequences of instructions further include instructions to deliver a plurality of pacing pulses configured to pace a heart of the patient according to a predetermined cardiac rhythm. The sequences of instructions further include instructions to determine the predetermined one or more times based on the one or more timing parameters and/or one or more morphology parameters of the ECG signals and further based on the predetermined cardiac rhythm.

The instructions to apply the at least one or the series of cardiac rhythm disruptive shocks at the predetermined one or more times using the plurality of therapy electrodes of the wearable cardiac treatment system further include instructions to apply a first cardiac rhythm disruptive shock at a first energy level and detect that no cardiac rhythm change has occurred in the patient. The instructions to apply the at least one or the series of cardiac rhythm disruptive shocks at the predetermined one or more times using the plurality of therapy electrodes of the wearable cardiac treatment system further include instructions to apply a second cardiac rhythm disruptive shock at a second energy level lower than the first energy level. The instructions to apply the at least one or the series of cardiac rhythm disruptive shocks at the predetermined one or more times using the plurality of therapy electrodes of the wearable cardiac treatment system further include instructions to apply a first cardiac rhythm disruptive shock at a first energy level, the first cardiac rhythm disruptive shock inducing the cardiac rhythm change in the patient. The sequences of instructions further include instructions to apply, using the plurality of therapy electrodes, a second cardiac rhythm disruptive shock at a second energy level higher than the first energy level.

The sequences of instructions further include instructions to apply, using the plurality of therapy electrodes, a cardiac rhythm restoring shock to the patient to restore a normal cardiac rhythm on detecting the cardiac rhythm change in the patient. The cardiac rhythm restoring shock includes a defibrillation shock. The instructions to apply, using the plurality of therapy electrodes, the cardiac rhythm restoring shock further include instructions to apply a first cardiac rhythm restoring shock at a first restoring shock energy level, detect that the patient has not been restored to the normal cardiac rhythm, and apply a second cardiac rhythm restoring shock at a second restoring shock energy level higher than the first restoring shock energy level. The sequences of instructions further include instructions to record, in the memory, a restoring shock energy level of the cardiac rhythm restoring shock that restored the patient to the normal cardiac rhythm. The instructions to adjust the defibrillation energy level for the one or more future defibrillation shocks to be delivered by the wearable cardiac treatment system further include instructions to adjust the defibrillation energy level for the one or more future defibrillation shocks to be delivered by the wearable cardiac treatment system based on the energy level of the cardiac rhythm disruptive shock that induced the cardiac rhythm change and further based on the restoring shock energy level of the cardiac rhythm restoring shock that restored the patient to the normal cardiac rhythm.

The sequences of instructions further include instructions to repeat applying the at least one or the series of cardiac rhythm disruptive shocks, detecting the cardiac rhythm change in the patient, and recording the energy level of the cardiac rhythm disruptive shock that induced the cardiac rhythm change. The sequences of instructions further include instructions to construct a dose-response curve for the patient based on the recorded energy levels of the cardiac rhythm disruptive shocks that induced the cardiac rhythm changes. The sequences of instructions further include instructions to determine a predetermined percentile of the dose-response curve. The predetermined percentile includes a 50th percentile. The sequences of instructions to adjust the defibrillation energy level for the one or more future defibrillation shocks to be delivered by the wearable cardiac treatment system further include instructions to adjust the defibrillation energy level for the one or more future defibrillation shocks to be delivered by the wearable cardiac treatment system using an energy level corresponding to the predetermined percentile of the dose-response curve. The instructions to adjust the defibrillation energy level for the one or more future defibrillation shocks to be delivered by the wearable cardiac treatment system further include instructions to perform a statistical analysis on the energy levels of the cardiac rhythm disruptive shocks that induced the cardiac rhythm changes and adjust the defibrillation energy level for the one or more future defibrillation shocks based on the statistical analysis.

According to an aspect of the disclosure we provide a cardiac treatment system configured to treat arrhythmias occurring in an patient, comprising: a plurality of ECG electrodes configured to sense ECG signals indicative of cardiac activity in the patient; a plurality of therapy electrodes; a memory configured to store baseline therapy energy information; and a cardiac controller comprising one or more processors in communication with the memory. In one or more examples, the plurality of ECG electrodes, and the plurality of therapy electrodes, the one or more processors are configured to provide one or more processes for determination of a deviation from energy levels specified by the baseline therapy energy information.

### BRIEF DESCRIPTION OF THE DRAWINGS

Various aspects of at least one example are discussed below with reference to the accompanying figures, which are not intended to be drawn to scale. The figures are included to provide an illustration and a further understanding of the various aspects and examples, and are incorporated in and constitute a part of this specification, but are not intended to limit the scope of the disclosure. The drawings, together with the remainder of the specification, serve to explain principles and operations of the described and claimed aspects and examples. In the figures, each identical or nearly identical component that is illustrated in various figures is represented by a like numeral. For purposes of clarity, not every component may be labeled in every figure.

FIG. 1 depicts an example setting where a baselining session may be performed for a patient prescribed to wear a wearable cardiac treatment system.

FIG. 2 depicts an example wearable cardiac treatment system.

FIG. 3 depicts an example wearable cardiac treatment device.

FIG. 4 depicts an example sample process flow for performing a baseline therapy energy session.

FIG. 5 depicts another example sample process flow for performing a baseline therapy energy session.

FIG. 6A depicts an example electronic architecture for a wearable cardiac treatment device.

FIG. 6B depicts another example electronic architecture for a wearable cardiac treatment device.

FIG. 7 depicts an example dose-response curve.

FIG. 8 depicts an example cardiac rhythm disruptive shock schedule.

FIG. 9 depicts another example dose-response curve.

FIG. 10 depicts another example wearable cardiac treatment device.

FIG. 11 depicts another example wearable cardiac treatment device.

### DETAILED DESCRIPTION

Wearable medical devices, such as wearable cardiac treatment devices, are used in clinical, outpatient, or in-hospital (inpatient) care settings to monitor for treatable cardiac arrhythmias and provide treatment such as defibrillation, cardioversion, or pacing shocks in the event of life-threatening arrhythmias. In examples, clinical settings include a broad array of medical service providers and places where healthcare occurs, including urgent care centers, rehabilitation centers, nursing homes, and long-term care facilities. In examples, outpatient care settings include settings where medical procedures, tests, and/or monitoring services are provided to patients without being admitted to a hospital, e.g., such as for an overnight hospital stay. Outpatient settings can include cardiology clinics, testing centers, providers of medical procedures on an outpatient basis, wellness and prevention services at outpatient clinics, rehabilitation centers, specialized outpatient service providers (e.g., hemodialysis, chemotherapy, etc.) or other similar care providers, and/or outpatient cardiac counseling program administrators or providers. Ambulatory patients in such clinical and/or outpatient settings can be prescribed a wearable defibrillator or a wearable cardioverter defibrillator (WCD). In-hospital care settings, on the other hand, include settings where medical procedures, tests, and/or monitoring services are provided to a patient on admission to a hospital, e.g., for an overnight hospital stay. Such in-hospital or inpatient care settings include emergency room (ER) visits and stays, intensive care unit (ICU) stays, or settings where patients are admitted to stay for a period of time (e.g., overnight), whether briefly or for an extended period of time. Patients in such in-hospital or inpatient settings can be prescribed a hospital wearable defibrillator (HWD), also described in further detail below.

A wearable cardiac treatment device, such as a WCD or an HWD, includes therapy electrodes or defibrillator pads positioned on an upper torso of a patient. In the case of a WCD, the therapy electrodes are disposed within a garment worn about the upper torso of the patient as described in further detail below. In the case of an HWD, the therapy electrodes are disposed within pads that are adhesively attached to the upper torso of the patient. The device is configured to continuously monitor the patient's heart to detect the heart rhythm. In the event a lethal cardiac arrhythmia is detected, the device can provide the patient with predetermined alarms, e.g., a vibration and/or gong alert that indicates the patient's attention is required and that a therapeutic shock is imminent. The patient can respond to the alarms by pressing buttons or otherwise providing a response to the device to cause the device to suspend the shock. If the patient does not respond to the alarms within a certain period of time (e.g., 45 seconds to about 75 seconds), the device is configured to deliver the therapeutic shock, e.g., a defibrillation shock. The device can be configured to deliver multiple shocks in this manner so long as underlying cardiac signals indicate an ongoing arrhythmia condition in the patient.

In such wearable cardiac treatment devices, the energy level for defibrillation shocks must be set high enough such that the shocks are effective to treat potentially lethal cardiac arrhythmias occurring in a patient. In implementations, the amount of energy of defibrillation is a variable that can determine defibrillation success. For example, a relationship between defibrillation success and defibrillation energy can be a logistic relationship. Such a logistic relationship resembles a “dose-response” curve where lower energies are likely to be less successful and higher energies are more likely to be more successful. The threshold at which a predetermined amount of defibrillation energy succeeds in converting an underlying cardiac arrhythmia a certain percentage of times is known as the defibrillation threshold (“DFT”). For example, the “DFT50” and “DFT90” represent energies where defibrillations succeed in converting an underlying cardiac arrhythmia 50% and 90% of the time, respectively. At the same time, defibrillation shocks require a certain amount of energy from the components of wearable cardiac treatment devices (e.g., capacitors of wearable cardiac treatment devices), and consequently have to be sized appropriately, which impacts weight and mobility. Additionally, the larger the energy level of a defibrillation shock, the more painful the defibrillation shock may be for the patient (e.g., the more likely it is the defibrillation shock may burn the skin of the patient). As such, given that the dose-response curve is different for each patient, it would be beneficial for wearable cardiac treatment devices to be configured to determine an effective energy level for defibrillation shocks on a patient-by-patient basis. In implementations, a DFT dose-response curve can be determined for the patient through a baseline therapy energy process, and a defibrillation energy customized to the patient, to help avoid negative device and patient consequences noted above.

The baseline therapy energy sessions described herein involve delivering a plurality of shocks to a patient in normal sinus rhythm to induce detectable cardiac rhythm changes in the patient. The energy levels used to successfully induce a cardiac rhythm change can be plotted to determine one or more dose-response curves for the patient. For example, the dose-response curve for a patient may show the probability that a shock at a given energy level will not induce ventricular fibrillation in the patient. In examples, these one or more dose-response curves can be used to estimate the DFT. As an illustration, the patient's dose-response 50th percentile (e.g., a shock energy level that induces a cardiac rhythm change in the patient 50% of the time) may approximate the DFT90 (e.g., a shock energy level that successfully treats a cardiac arrhythmia being experienced by the patient 90% of the time). As another illustration, the energy level used to successfully induce the cardiac rhythm change may be increased by a predetermined amount (e.g., 3 Joules, 5 Joules, 10 Joules, doubled, or other user-configurable energy) to estimate the DFT90.

As such, this disclosure relates to a wearable cardiac treatment system configured to treat arrhythmias occurring in an ambulatory patient, where the wearable cardiac treatment system is configured to perform a baseline therapy energy session for the patient before providing defibrillation shocks to treat arrhythmias occurring in the patient. A patient is prescribed a wearable cardiac treatment device configured to continuously monitor an ambulatory cardiac patient for arrhythmias. The wearable cardiac treatment device includes a garment configured to be worn about a torso of the patient, ECG electrodes configured to be disposed on the garment and further configured to sense ECG signals indicative of cardiac activity in the patient, therapy electrodes configured to be disposed on the garment, and a cardiac controller. The cardiac controller includes one or more processors in communication with a memory disposed, for example, in the cardiac controller. In examples, the cardiac controller is in communication with a remote server via a network. In implementations, cardiac controller processors may execute the baseline therapy energy process described herein without connecting to a remote server and store the results in a memory device on the controller. The cardiac controller may then be configured to provide defibrillation or cardioversion pulses based on the output of the baseline therapy energy process.

In some implementations, the cardiac controller can implement the baseline therapy energy process in cooperation with the remote server. For example, the cardiac controller can transmit ECG information collected from the patient to the remote server, and the remote server can implement the baselining process described herein. In such an implementation, the remote server can additionally (or alternatively) transmit instructions or messages to the on-site technician, caregiver and/or patient based on output of the baselining therapy energy process described herein. Such instructions or messages can be displayed on a user interface device or output via a speaker on the cardiac controller. For example, such messages may include results of the baselining therapy session that the technician or caregiver can use to modify future defibrillation energies for the patient.

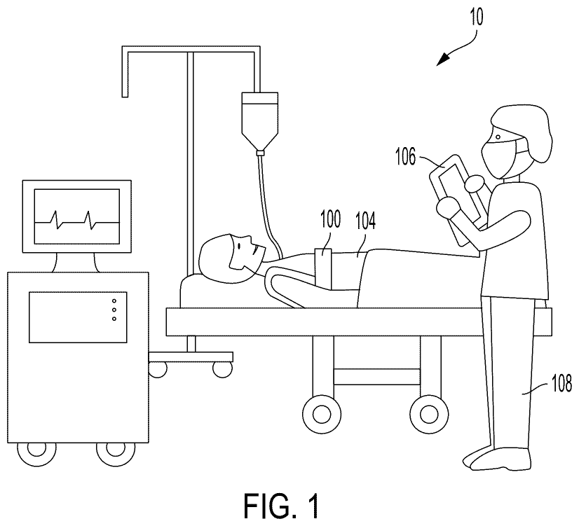

Once the patient is prescribed the wearable cardiac treatment device in a clinical, outpatient, or in-hospital setting, a baselining therapy energy session is performed with the patient. FIG. 1 illustrates an example of an in-hospital setting 10 in which a baselining therapy energy session can be performed. As shown in FIG. 1, a patient 104 is wearing a wearable cardiac treatment device 100 and undergoing the baselining therapy session under a technician 108. As another example, a technician may perform the baselining therapy energy session with the patient under the care and supervision of the patient's caregiver (e.g., the patient's prescribing physician). In some examples, the patient may be sedated during the baselining therapy energy session, e.g., as shown by the patient 104 in FIG. 1. The patient may be sedated specifically for the baselining therapy energy session, or the patient may have been sedated due to other medical procedures. For instance, the patient may have experienced a severe cardiac event, such as a myocardial infarction, and have been sedated related to procedures used to treat the severe cardiac event. The baselining therapy energy session may thus be performed with the patient while the patient is still sedated. In some examples, the patient may be awake for the baselining therapy energy session (e.g., depending on the type of desired cardiac rhythm change to be induced in the patient, as described in further detail below).

During the baselining therapy energy session, the cardiac controller is configured to determine timing parameters and/or morphology parameters of ECG signals sensed by the ECG electrodes of the wearable cardiac treatment device. For example, the timing parameters and/or morphology parameters may correspond with various parts of the ECG waveform, such as the T-waves or R-waves. Once the cardiac controller has determined the timing parameters and/or morphology parameters, the cardiac controller applies cardiac rhythm disruptive shocks to the patient via the therapy electrodes at predetermined one or more times. These predetermined one or more times are based on the timing parameters and/or morphology parameters of the ECG signals. For instance, the predetermined one or more times may be based on or synchronized with timings for one or more T-waves occurring the in the patient, where the cardiac controller determines the timings for the T-waves using the timing parameters and/or morphology parameters.

The cardiac controller applies cardiac rhythm disruptive shocks at same and/or decreasing energy levels until a cardiac rhythm change is induced in the patient. For example, the cardiac controller may apply cardiac rhythm disruptive shocks to the patient until the patient experiences a ventricular fibrillation (VF) rhythm or a premature ventricular contraction (PVC). The cardiac controller records an energy level of a cardiac rhythm disruptive shock that induced the cardiac rhythm change in the memory as baseline therapy energy information. The controller may record other parameters of the cardiac rhythm disruptive shock, e.g., a morphology, tilt, slope, or other similar parameters.

The cardiac controller also adjusts a defibrillation energy level for future defibrillation shocks based on the energy level of the cardiac rhythm disruptive shock that induced the cardiac rhythm change. The cardiac controller may use the energy level as an input to an algorithm or look-up table that correlates energy levels for cardiac rhythm disruptive shocks that induced a cardiac rhythm change with successful defibrillation energy levels for treating cardiac arrhythmias. The cardiac controller may then set the output of the algorithm or look-up table as the defibrillation energy level for future defibrillation shocks. In one example, such an algorithm or look-up table may be based on a dose-response curve as described herein. Other examples are described in detail below. Accordingly, a baseline therapy energy process implemented in the cardiac controller or a remote server is configured to determine a likely successful defibrillation energy level based on the energy level of the cardiac rhythm disruptive shock that induced the cardiac rhythm change and a dose-response curve for the patient. The baseline therapy energy process implemented in the cardiac controller or a remote server is configured to cause the cardiac controller to set the defibrillation energy level for future defibrillation shocks based on the energy level of the cardiac rhythm disruptive shock that induced the cardiac rhythm change.

As an illustration of the baseline therapy energy process, the cardiac controller may determine timing parameters and/or morphology parameters of T-waves in the patient based on the sensed ECG signals. The cardiac controller may apply fibrillation shocks at predetermined times based on the timing parameters and/or morphology parameters of the T-waves, where the cardiac controller continues applying fibrillation shocks at same and/or decreasing energy levels until the patient enters a ventricular fibrillation state. The cardiac controller detects that the patient has entered the fibrillation state and applies, via the therapy electrodes, a defibrillation shock to the patient to treat the ventricular fibrillation state. The cardiac controller then records the energy level of the fibrillation shock that induced the ventricular fibrillation state as baseline therapy energy information and adjusts a defibrillation energy level for future defibrillation shocks based on the energy level of the fibrillation shock that induced the ventricular fibrillation state.

In one example use case, a clinician or other caregiver prescribes that a patient at risk of heart failure wear a wearable cardiac treatment device for a certain amount of time (e.g., until the patient is scheduled for a surgery to receive an implantable cardiac defibrillator). A technician fits the patient for a wearable cardiac treatment device and performs a baselining therapy energy session with the patient while the patient is sedated. For instance, the patient may be sedated for the baselining therapy energy session, or the patient may have already been sedated due to a medical procedure the patient is undergoing (e.g., related to a severe cardiac event that prompted the prescription for the wearable cardiac treatment device). The wearable cardiac treatment device applies fibrillation shocks to the patient on or in the proximity of the T-waves of the patient at decreasing energy levels until the patient enters a ventricular fibrillation state, after which the wearable cardiac treatment device applies a defibrillation shock to the patient to treat the fibrillation state. For example, a typical T-wave duration is around 0.05 seconds (50 milliseconds) to around 0.30 seconds (300 milliseconds). For example, applying the fibrillation shocks to within a proximity of the T-waves includes applying the fibrillation shocks to predetermined periods of times prior to or immediately after a certain preidentified fiducial point on a T-wave. For example, a predetermined local maximum can be determined as the preidentified fiducial point, and the proximity of the T-wave can be set to be within 0.001 to 1 microsecond, within 1 to 10 microseconds, within 10 to 100 microseconds, within 100 to 10 microseconds, etc. from the preidentified fiducial point (prior to or immediately after the preidentified fiducial point). In implementations, the technician, clinician, or caregiver can set the proximity via user-configurable parameters displayed or provided on a user interface of the cardiac controller. The wearable cardiac treatment device also repeats this process of applying fibrillation shocks to the patient until the patient enters a ventricular fibrillation state a certain number of times (e.g., three times, five times, eight times, or other predetermined number of times).

The wearable cardiac treatment device records the energy levels of the fibrillation shocks that induced the ventricular fibrillation state, as well as the energy levels of the defibrillation shocks that successfully treated the ventricular fibrillation state. The wearable cardiac treatment device transmits these energy levels to a remote server, which uses the fibrillation and defibrillation energy levels identified in the baseline therapy energy session to determine a defibrillation energy level likely to be effective (e.g., successful in converting a cardiac arrhythmia) for the patient at a predetermined success rate, e.g., 90%, of the time, 95% of the time, or other such success rate as may be deemed by the technician or prescribing physician. The remote server then transmits the defibrillation energy level to the wearable cardiac treatment device. The wearable cardiac treatment device stores the defibrillation energy level received from the remote server for use in the future should the wearable cardiac treatment device determine that the patient is experiencing a treatable cardiac arrhythmia.

In another example use case, a clinician or other caregiver prescribes that a patient at risk of heart failure wear a wearable cardiac treatment device for an extended period of time because surgery to receive an implantable cardiac defibrillator is too risky for the patient. A technician fits the patient for a wearable cardiac treatment device and performs a baselining therapy energy session with the patient while the patient is awake. The wearable cardiac treatment device applies cardiac rhythm disruptive shocks to the patient at a first energy level (e.g., pacing shocks asynchronous with the patient's normal cardiac rhythm) and determines whether the patient has experienced a premature ventricular contraction. The wearable cardiac treatment device then records the first energy level, as well as data corresponding to the patient's return to their normal cardiac rhythm (e.g., how many beats it takes for the patient to return to a normal cardiac rhythm, delays between successive beats as the patient returns to a normal cardiac rhythm, etc.). If the patient has not experienced a premature ventricular contraction, the wearable cardiac treatment device applies additional cardiac rhythm disruptive shocks to the patient (e.g., at a second, lower energy level; at a third, higher energy level; according to a different timing asynchronous to the patient's normal cardiac rhythm; etc.) and determines whether the patient has experienced a premature ventricular contraction. The wearable cardiac treatment device repeats the process of modifying the energy level, such as lowering the energy level, for cardiac rhythm disruptive shocks until the patient experiences a premature ventricular contraction.

The wearable cardiac treatment device uses the data recorded from this baselining therapy session to determine a defibrillation energy level likely to be effective (e.g., successful in converting a cardiac arrhythmia) for the patient at a predetermined success rate, e.g., 90% of the time, 95% of the time, or other such success rate as may be deemed by the technician or prescribing physician. The wearable cardiac treatment device then uses this defibrillation energy level to treat cardiac arrhythmias occurring in the patient (e.g., types of cardiac arrhythmias treatable by a defibrillation shock, such as VF). As an example, the wearable cardiac treatment device uses the energy level that successfully caused a premature ventricular contraction and/or uses the data corresponding to the patient's return to their normal cardiac rhythm to determine the defibrillation energy level likely to be effective. Using the data from the baselining therapy energy session, the wearable cardiac treatment device is configured to determine a delay period between detecting a treatable cardiac arrhythmia and applying the defibrillation shock at the defibrillation energy level to be used for these future defibrillation shocks to treat cardiac arrhythmias occurring in the patient.

The wearable cardiac treatment system described herein may provide several advantages over prior art systems. The techniques described with respect to the wearable cardiac treatment system allow a wearable cardiac treatment device to provide a programmed, patient-specific defibrillation energy that could be delivered at an energy lower than the generic defibrillation energy delivered by the wearable cardiac treatment device. Providing this lower defibrillation energy option may allow for lighter hardware for the wearable cardiac treatment device, as the wearable cardiac treatment device may not need as large of capacitors to provide the lower defibrillation energy level to the patient. As such, if the baseline therapy energy session identifies that the patient can be effectively treated with a defibrillation energy level below a certain threshold, a technician may provide the patient with a lighter, more comfortable wearable cardiac treatment device.

Additionally, defibrillation at lower energies would provide for a lower risk of post-defibrillation myocardial dysfunction. A lower defibrillation energy level may also decrease the chance, for example, that the defibrillation shock will burn the skin of the patient during defibrillation. As such, performing a baseline therapy energy session to potentially identify a lower defibrillation energy level for a patient that will still likely be effective may also lead to better health outcomes for the patient after a defibrillation.

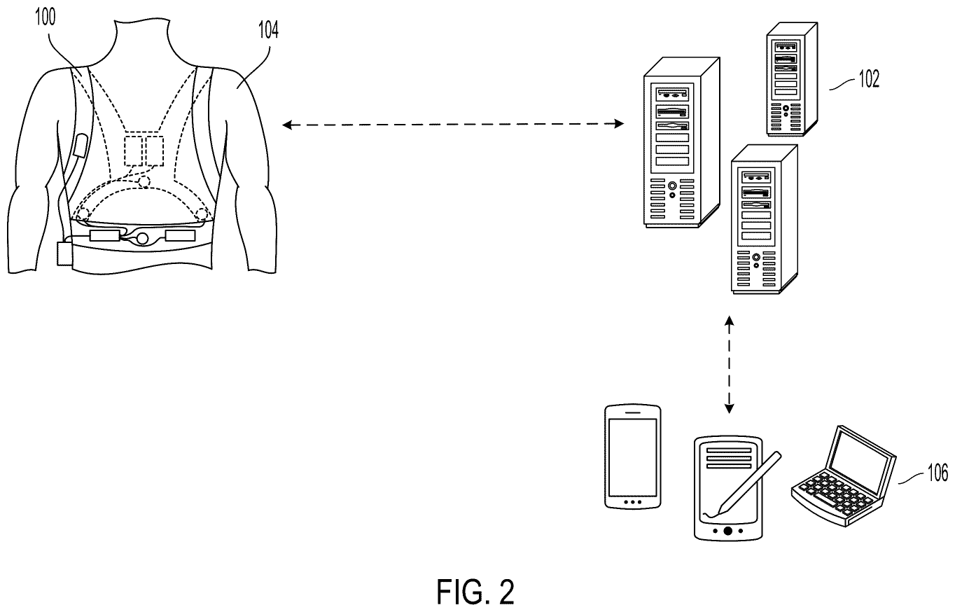

FIG. 2 shows a wearable cardiac treatment system that includes a wearable cardiac treatment device 100 in communication with a remote server 102. The wearable cardiac treatment device 100 is configured to treat arrhythmias occurring in an ambulatory patient 104. In implementations, the wearable cardiac treatment device 100 may be implemented as a wearable garment configured to be worn about a torso of the patient 104. The wearable garment may be further configured to be worn continuously by the patient 104 for an extended period of time. Additionally, the wearable cardiac treatment device 100 may include a plurality of ECG electrodes configured to be disposed on the garment and further configured to sense ECG signals indicative of cardiac activity in the patient, as well as a plurality of therapy electrodes configured to be disposed on the garment. In implementations, the wearable cardiac treatment device 100 may include one or more other externally worn sensors configured to be disposed on the garment and output one or more physiological signals for the patient 104 and/or for the environment of the patient 104, such as vibrational sensors (e.g., biovibrational sensors configured to detect heart sounds), photoplethysmography sensors, radiofrequency (RF) sensors (which may be used, for example, to determine lung fluid content in the patient 104), temperature sensors, humidity sensors, and/or the like. The wearable cardiac treatment device 100 may further include a plurality of treatment electrodes configured to deliver shocks to the patient 104, which may include cardiac disruptive shocks, cardiac rhythm restoring shocks, pacing pulses, and/or the like.

The wearable cardiac treatment device 100 is configured to transmit signals and data generated by the wearable cardiac treatment device 100 to the remote server 102. Accordingly, the wearable cardiac treatment device 100 may be in wireless communication with the remote server 102. As an illustration, the wearable cardiac treatment device 100 may communicate with the remote server 102 via cellular networks, via Bluetooth®-to-TCP/IP access point communication, via Wi-Fi, and the like. As such, the wearable cardiac treatment device 100 may include communications circuitry configured to implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the communications circuitry in the wearable cardiac treatment device 100 may be part of an Internet of Things (IoT) and communicate with the remote server 102 via IoT protocols (e.g., Constrained Application Protocol (CoAP), Message Queuing Telemetry Transport (MQTT), Wi-Fi, Zigbee, Bluetooth®, Extensible Messaging and Presence Protocol (XMPP), Data-Distribution Service (DDS), Advanced Messaging Queuing Protocol (AMQP), and/or Lightweight M2M (LwM2M)).

The remote server 102 is configured to receive and, in implementations, store and process the signals and data transmitted by the wearable cardiac treatment device 100 worn by the ambulatory patient 104. Accordingly, the remote server 102 may include a computing device, or a network of computing devices, including at least one database (e.g., implemented in non-transitory computer-readable media or memory) and at least one processor configured to execute sequences of instructions (e.g., stored in the database, with the at least one processor being in communication with the database). The sequences of instructions may be configured to receive and process the signals transmitted by the wearable cardiac treatment device 100. For example, the at least one processor of the remote server 102 may be configured similarly to the processor 516 of the wearable cardiac treatment device 100 discussed in further detail below. The database may be implemented as flash memory, solid state memory, magnetic memory, optical memory, cache memory, combinations thereof, and/or others.

As further shown in FIG. 2, in implementations, the wearable cardiac treatment system may include one or more user interfaces, such as one or more clinician-authorized user terminals 106. The user terminals 106 are in electronic communication with the remote server 102 through a wired or wireless connection. For instance, the user terminals 106 may communicate with the remote server 102 via Wi-Fi, via Ethernet, via cellular networks, and/or the like. The user terminals 106 may include, for example, desktop computers, laptop computers, and/or portable personal digital assistants (e.g., smartphones, tablet computers, etc.).

The one or more clinician-authorized user terminals 106 are configured to electronically communicate with the remote server 102 for the purpose of sending and receiving information relating to the patient 104 wearing the wearable cardiac treatment device 100. In implementations, the user terminals 106 are configured to allow clinicians to view information on the patient 104 wearing the wearable cardiac treatment device 100. For example a user terminal 106 may display to the user (e.g., a clinician or other caregiver associated with the patient 104) information from a baselining therapy energy session conducted with the patient 104. In implementations, the user terminals 106 may display additional information about the wearable cardiac treatment device 100 and/or the patient 104, such as one or more reports summarizing arrhythmia information for the patient 104, health information for the patient 104 (e.g., activity information for the patient 104, sleep information for the patient 104), wear status information for the patient 104 (e.g., how many hours per day the patient 104 wears the wearable cardiac treatment device 100), and/or the like.

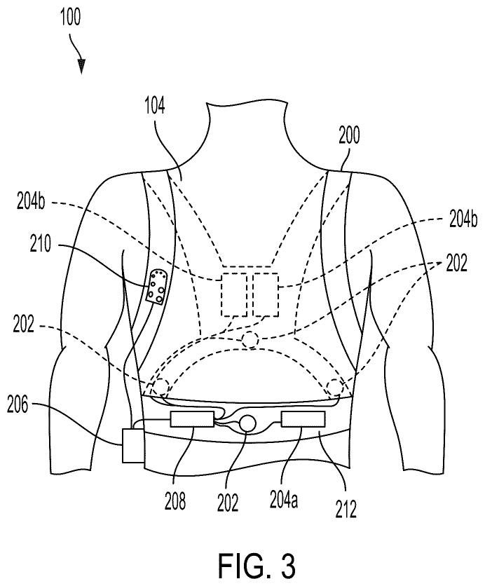

FIG. 3 shows the wearable cardiac treatment device 100, according to implementations. As shown in FIG. 3, the wearable cardiac treatment device 100 is external and wearable by the patient 104 around the patient's torso. Such a wearable cardiac treatment device 100 can be, for example, capable and designed for moving with the patient 104 as the patient 104 goes about their daily routine. For example, the wearable cardiac treatment device 100 may be configured to be bodily-attached to the patient 104. As noted above, the wearable cardiac treatment device 100 may be a wearable defibrillator or a wearable cardioverter defibrillator. In one example scenario, such wearable defibrillators can be worn nearly continuously or substantially continuously for a week, two weeks, a month, or two or three months at a time. During the period of time in which they are worn by the patient 104, the wearable defibrillators can be configured to continuously or substantially continuously monitor the vital signs of the patient 104 and can be configured to, upon determination that treatment is required, deliver one or more therapeutic electrical pulses to the patient 104. For example, such therapeutic shocks can be pacing, defibrillation, cardioversion, or transcutaneous electrical nerve stimulation (TENS) pulses.

As shown in FIG. 3, the wearable cardiac treatment device 100 can include one or more of the following: a garment 200 configured to be worn about the patient's torso, one or more ECG electrodes 202 configured to be disposed on the garment 200 and further configured to sense ECG signals indicative of cardiac activity in the patient 104, one or more therapy electrodes 204a and 204b (collectively referred to herein as therapy electrodes 204) configured to be disposed on the garment 200, a cardiac controller 206, a connection pod 208, a patient interface pod 210, a belt 212, or any combination of these. In implementations, the wearable cardiac treatment device 100 may also include additional sensors, such as one or more motion detectors configured to generate motion data indicative of physical activity performed by the patient 104, one or more wear state sensors configured to detect a wear state of the wearable cardiac treatment device 100, one or more vibrational or bioacoustics sensors configured to generate bioacoustics signals for the heart of the patient 104, one or more respiration sensors configured to generate respiration signals indicative of the respiration activity of the patient 104, and/or the like.

In examples, at least some of the components of the wearable cardiac treatment device 100 can be configured to be disposed on the garment 200 by being removably mounted on or affixed to the garment 200, such as by mating hooks, hook-and-loop fabric strips, receptacles (e.g., pockets), snaps (e.g., plastic or metal snaps), and the like. For instance, the ECG electrodes 202 may be removably attached to the garment 200 by hook-and-loop fabric strips on the ECG electrodes 202 and the garment 200, and the therapy electrodes 204 may be removably attached on the garment 200 by being inserted into receptacles of the garment 200. In some examples, at least some of the components of the wearable cardiac treatment device can be permanently integrated into the garment 200, such as by being sewn into the garment or by being adhesively secured to the garment 200 with a permanent adhesive. In examples, at least some of the components may be connected to each other through cables, through sewn-in connections (e.g., wires woven into the fabric of the garment 200), through conductive fabric of the garment 200, and/or the like.

The cardiac controller 206 can be operatively coupled to the ECG electrodes 202 and the therapy electrodes 204, which can be temporarily or removably affixed to the garment 200 (e.g., assembled into the garment 200 or removably attached to the garment 200, for example, using hook-and-loop fasteners) and/or permanently integrated into the garment 200 as discussed above. As shown in FIG. 3, the ECG electrodes 202 and/or the therapy electrodes 204 can be directly operatively coupled to the cardiac controller 206 and/or operatively coupled to the cardiac controller 206 through the connection pod 208. Component configurations other than those shown in FIG. 3 are also possible. For example, the ECG electrodes 202 can be configured to be attached at various positions about the body of the patient 104. In some implementations, the ECG electrodes 202 and/or at least one of the therapy electrodes 204 can be included on a single integrated patch and adhesively applied to the patient's body. In some implementations, the ECG electrodes 202 and/or at least one of the therapy electrodes 204 can be included in multiple patches and adhesively applied to the patient's body. Such patches may be in a wired (e.g., via the connection pod 208) or wireless connection with the cardiac controller 206.

As discussed above, the ECG electrodes 202 can be configured to detect ECG signals indicative of cardiac activity of the patient 104. Example ECG electrodes 202 may include a metal electrode with an oxide coating such as tantalum pentoxide electrodes. For example, by design, the ECG electrodes 202 can include skin-contacting electrode surfaces that may be deemed polarizable or non-polarizable depending on a variety of factors including the metals and/or coatings used in constructing the electrode surface. All such electrodes can be used with the principles, techniques, devices and systems described herein. For example, the electrode surfaces can be based on stainless steel, noble metals such as platinum, or Ag—AgCl.

In implementations, the ECG electrodes 202 can be used with an electrolytic gel dispersed between the electrode surface and the patient's skin. In implementations, the ECG electrodes 202 can be dry electrodes that do not need an electrolytic material. As an example, such a dry electrode can be based on tantalum metal and having a tantalum pentoxide coating as is described above. Such dry electrodes can be more comfortable for long term monitoring applications.

In implementations, the ECG electrodes 202 can include additional components such as accelerometers, acoustic signal detecting devices (e.g., vibrational sensors), and other measuring devices for recording additional parameters. For example, the ECG electrodes 202 can also be configured to detect other types of patient physiological parameters and acoustic signals, such as tissue fluid levels, heart vibrations, lung vibrations, respiration vibrations, patient movement, etc. In implementations, the wearable cardiac treatment device 100 may include sensors or detectors separate from the ECG electrodes 202, such as separate motion detector(s), wear state detector(s), vibrational sensor(s), bioacoustics sensor(s), respiration sensor(s), temperature sensor(s), pressure sensor(s), and/or the like. In some examples, the therapy electrodes 204 can also be configured to include sensors configured to detect ECG signals as well as, or in the alternative, other physiological signals from the patient 104.

The connection pod 208 can, in some examples, include a signal processor configured to amplify, filter, and digitize cardiac signals, such as the ECG signals, prior to transmitting the cardiac signals to the cardiac controller 206. One or more therapy electrodes 204 can be configured to deliver one or more therapeutic cardioversion/defibrillation shocks to the body of the patient 104 when the wearable cardiac treatment device 100 determines that such treatment is warranted based on the signals detected by the ECG electrodes 202 and processed by the cardiac controller 206. Example therapy electrodes 204 can include conductive metal electrodes such as stainless-steel electrodes that include, in certain implementations, one or more conductive gel deployment devices configured to deliver conductive gel between the metal electrode and the patient's skin prior to delivery of a therapeutic shock.

In implementations, the cardiac controller 206 may also be configured to warn the patient 104 prior to the delivery of a therapeutic shock, such as via output devices integrated into or connected to the cardiac controller 206, the connection pod 208, and/or the patient interface pod 210. The warning may be auditory (e.g., a siren alarm, a voice instruction indicating that the patient 104 is going to be shocked), visual (e.g., flashing lights on the cardiac controller 206), haptic (e.g., a tactile, buzzing alarm generated by the connection pod 208), and/or the like. If the patient 104 is still conscious, the patient 104 may be able to delay or stop the delivery of the therapeutic shock. For example, the patient 104 may press one or more buttons on the patient interface pod 210 to indicate that the patient 104 is still conscious. In response to the patient 104 pushing the one or more buttons, the cardiac controller 206 may delay or stop the delivery of the therapeutic shock.

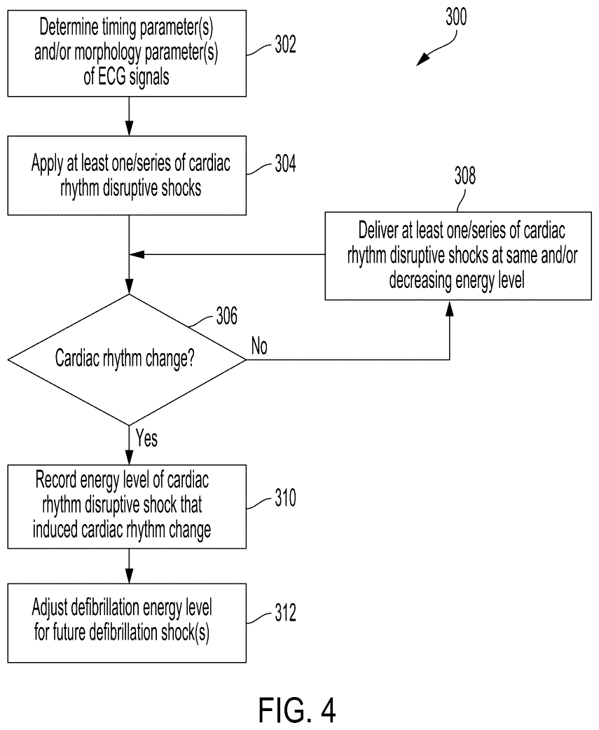

FIG. 4 illustrates a sample process flow for performing a baseline therapy energy session. The sample process 300 shown in FIG. 4 can be implemented by the cardiac controller 206. As shown in FIG. 4, the cardiac controller 206 determines timing parameters and/or morphology parameters of ECG signals at step 302. In implementations, the cardiac controller 206 analyzes the ECG signals to identify one or more parts of ECG waveforms (e.g., using ECG waveform templates stored in the data storage 502 of the cardiac controller 206, discussed below). For example, the cardiac controller 206 may identify P-waves, PQ segments, Q-waves, R-waves, S-waves, QRS complexes, ST segments, T-waves, and/or PR intervals of ECG waveforms. Using the one or more identified parts of the ECG waveforms, the cardiac controller 206 may determine one or more timing parameters and/or one or more morphology parameters of the ECG signal. For example, the cardiac controller 206 may identify morphologies of the ECG signal corresponding to T-waves and use the identified morphologies to predict the timings of one or more upcoming T-waves. As another example, the cardiac controller 206 may identify morphologies of the ECG signal corresponding to R-waves and predict the timings of one or more subsequent T-waves using the timings of the R-waves (e.g., by predicting that a T-waves occur a certain amount of time, such as 100-300 ms, after a peak of a given R-wave). As another example, the cardiac controller 206 may identify morphologies of T-waves as they occur. The identified one or more timing parameters and/or one or more morphology parameters of the ECG signal may depend on a desired type of cardiac rhythm change to induce in the patient 104. For instance, if the desired cardiac rhythm change is a ventricular fibrillation, the one or more timing parameters and/or one or more morphology parameters of the ECG signals may correspond to one or more T-waves in the patient 104, as discussed above, because applying one or more shocks to the patient 104 at the T-waves may induce a ventricular fibrillation in the patient 104. However, other types of cardiac rhythm changes in the patient 104 may be desired, such as a premature ventricular contraction or ventricular tachycardia.

In implementations, the cardiac controller 206 may be configured to deliver a series of pacing pulses configured to pace the heart of the patient 104 according to a predetermined cardiac rhythm during the baseline therapy energy session. Pacing the heart according to a predetermined cardiac rhythm may help the cardiac controller 206 determine one or more timings for the cardiac rhythm disruptive shocks, as discussed below. The cardiac controller 206 may deliver these pulses to the patient 104 via the therapy electrodes 204. For example, a maximum current level of a current waveform used to pace the heart of the patient 104 may range from 0 mAmps to about 200 mAmps. In some examples, a pulse width can range from about 0.05 ms to about 2 ms. In some examples, a frequency of the pulses can range from about 30 pulses per minute (PPM) to about 200 PPM. In accordance with one implementation, a 40 ms square wave pulse can be used. Examples of pacing current waveforms include a 40 ms constant current pulse, a 5 ms constant current pulse, and a variable current pulse. Additional details on providing pacing pulses to a patient using an external wearable defibrillator may be found in U.S. Pat. No. 8,983,597, filed on May 31, 2013, entitled “Medical Monitoring and Treatment Device with External Pacing,” which is hereby incorporated by reference. Accordingly, in implementations, the cardiac controller 206 may use the one or more timing parameters and/or one or more morphology parameters to determine when to deliver pacing pulses to the patient 104.

The cardiac controller 206 applies at least one or a series of cardiac rhythm disruptive shocks at predetermined one or more times at step 304. For example, the cardiac controller 206 may apply a single shock to the patient 104 to attempt to induce a cardiac rhythm change in the patient 104, or the cardiac controller 206 may apply multiple shocks to the patient 104 to attempt to induce a cardiac rhythm change in the patient 104. In implementations, the cardiac rhythm disruptive shock(s) can be defibrillation-like shock(s) configured to induce an episode of ventricular fibrillation, pacing pulse(s) delivered asynchronously with the patient's normal cardiac rhythm to induce a premature ventricular contraction, and/or the like.

In implementations, the cardiac controller 206 determines the predetermined one or more times to apply the cardiac rhythm disruptive shocks based on the one or more timing parameters and/or one or more morphology parameters of the ECG signals of the patient 104. For example, the predetermined one or more times may be predicted timings for one or more upcoming T-waves in the patient 104, which are predicted using the one or more timing parameters and/or one or more morphology parameters as discussed above. In implementations where the cardiac controller 206 delivers pacing pulses to pace the heart of the patient 104 according to a predetermined cardiac rhythm, the cardiac controller 206 may determine the predetermined one or more times further based on the predetermined cardiac rhythm. As an illustration, the cardiac controller 206 may predict timings for one or more upcoming T-waves in the patient 104 based on known timings of R-waves in the patient 104 from the pacing pulses. As another example, the predetermined one or more times may be predicted timings for pacing pulses that are asynchronous with the patient's normal cardiac rhythm (e.g., asynchronous with the beginning of the patient's P-waves or QRS complex in their ECG).

The cardiac controller 206 determines whether a cardiac rhythm change has occurred in the patient 104 at step 306. In implementations, the cardiac controller 206 may identify whether a particular cardiac rhythm change has occurred in the patient, such as whether the patient 104 has entered ventricular fibrillation, whether the patient 104 has entered ventricular tachycardia, or whether the patient 104 has experienced a premature ventricular contraction. In implementations, the cardiac controller 206 may determine whether the patient 104 has experienced any type of cardiac rhythm change that is a deviation from the patient's normal cardiac sinus rhythm. If the cardiac controller 206 determines that the patient 104 has not experienced a cardiac rhythm change, the cardiac controller 206 may continue delivering the at least one or series of cardiac rhythm disruptive shocks at the same and/or at decreasing energy levels until a cardiac rhythm change is induced in the patient 104 (e.g., unless it is determined after application of a certain number of shocks that inducing a cardiac rhythm change in the patient 104 is unlikely, after which the baseline therapy energy session may be aborted or restarted). For example, if no cardiac rhythm change is induced in the patient 104, the cardiac controller 206 may immediately decrease the energy level of a subsequent cardiac rhythm disruptive shock (e.g., decrease the energy level by 3 Joules, 5 Joules, by 10 Joules, etc., or other user configurable energy). As another example, the cardiac controller 206 may deliver a certain number of cardiac rhythm disruptive shocks at the same energy level before decreasing the energy level of the cardiac rhythm disruptive shocks. As another example, the cardiac controller 206 may continue delivering the cardiac rhythm disruptive shocks at the same energy level until a cardiac rhythm change is induced in the patient 104 or the baseline therapy energy session is aborted.

However, once a cardiac rhythm change is detected in the patient 104, the cardiac controller 206 records the energy level of the cardiac rhythm disruptive shock (or shocks) that induced the cardiac rhythm change in the patient 104 as baseline therapy energy information in a memory of the wearable cardiac treatment system at step 310. In implementations, the cardiac controller 206 may alternatively, or additionally, record the energy level of the last cardiac rhythm disruptive shock (or shocks) that did not induce the cardiac rhythm change in the patient 104 as the baseline therapy energy information in the memory of the wearable cardiac treatment system. In implementations, the memory may be located in the cardiac controller 206. For example, the memory may be the data storage 502 discussed below. In implementations, the memory may be located in the remote server 102 (e.g., such that the wearable cardiac treatment device 100 may transmit the energy level of the cardiac rhythm disruptive shock that induced the cardiac rhythm change to the remote server 102 via a network).

In some examples, the cardiac controller 206 may apply a first cardiac rhythm disruptive shock at a first energy level, where the first cardiac rhythm disruptive shock does not induce a cardiac rhythm change in the patient 104. The cardiac controller 206 may thus detect that no cardiac rhythm change has occurred in the patient 104. Subsequently, the cardiac controller 206 may apply a second cardiac rhythm disruptive shock at a second energy level lower than the first energy level. For example, the first energy level may be an energy level around 70 to 90 Joules (e.g., where being around the range may including being within 1-5 Joules of this range, within 10% of this range, within 20% of this range, etc.), and the second energy level may be an energy level around 30 to 50 Joules. As another example, the first energy level may be an energy level from the range of around 20 to 90 Joules, and the second energy level may be a lower energy level from the range of around 20 to 90 Joules (e.g., 3 Joules, 5 Joules, or 10 Joules, or other user-configurable energy lower than the first energy level).

In some examples, as discussed in more detail below, the cardiac controller may repeat at least part of the process 300 (e.g., steps 304-310). As such, to illustrate, the cardiac controller 206 may apply a first cardiac rhythm disruptive shock at a first energy level, where the first cardiac rhythm disruptive shock induces a cardiac rhythm change in the patient 104. The cardiac controller 206 may then repeat the process 300 and, at step 304, start by applying a second cardiac rhythm disruptive shock at a second energy level higher than the first energy level to determine if applying a cardiac rhythm disruptive shock at the second energy level still induces a cardiac rhythm change in the patient 104. For example, the first energy level may be at an energy level from the range of around 20 to 90 Joules, and the second energy level may be a higher energy level from the range of around 20 to 90 Joules. As another example, the first energy level may be at an energy level around 20 to 90 Joules, and the second energy level may be an energy level around 70 to 90 Joules.

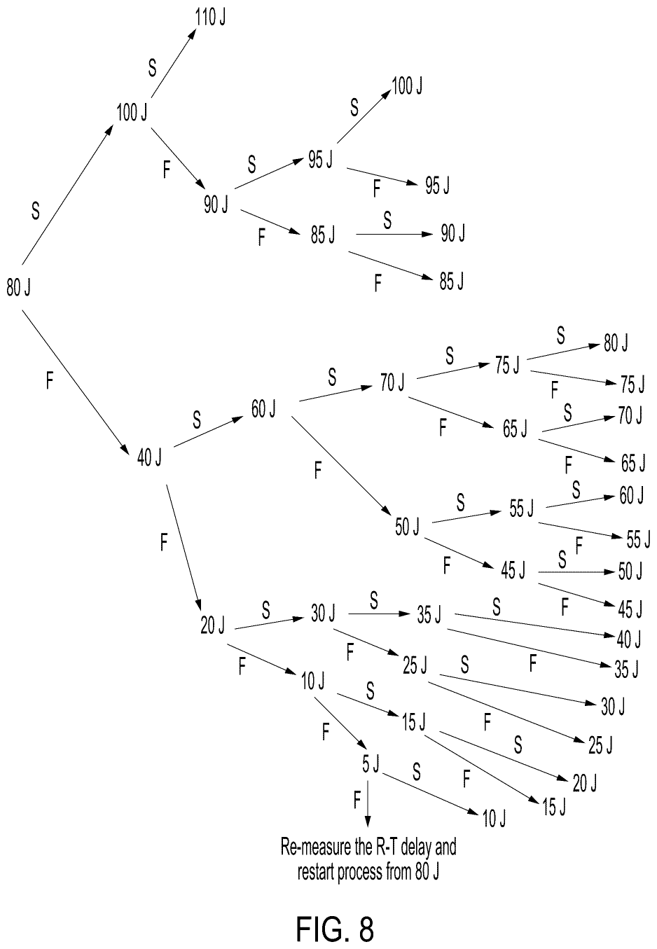

In implementations, the cardiac controller 206 repeat steps 304-310 of the process 300 according to a predetermined cardiac rhythm disruptive shock schedule. As an illustration, FIG. 8 shows an example cardiac rhythm disruptive shock schedule. As shown in FIG. 8, the cardiac controller 206 may apply a first cardiac rhythm disruptive shock at a first energy level (e.g., 80 J). If the first cardiac rhythm disruptive shock does not induce a cardiac rhythm change in the patient 104, the cardiac controller 206 may apply a second cardiac rhythm disruptive shock at a second energy level that is half of the first energy level (e.g., 40 J). If the second cardiac rhythm disruptive shock does not induce a cardiac rhythm change, the cardiac controller 206 may apply a third cardiac rhythm disruptive shock at a third energy level that is half of the second energy level (e.g., 20 J), and so on.

However, if the second cardiac rhythm disruptive shock does induce a cardiac rhythm change, when the cardiac controller 206 repeats the process 300, the cardiac controller 206 may apply a fourth cardiac rhythm disruptive shock at a fourth energy level that is higher than the second energy level by a predetermined amount (e.g., at 60 J). If the fourth cardiac rhythm disruptive shock does not induce a cardiac rhythm change, the cardiac controller 206 may apply a fifth cardiac rhythm disruptive shock at a fifth energy level that is lower than the fourth energy level by a predetermined amount (e.g., at 50 J), and so on until the cardiac controller 206 reaches one of the endpoints shown in FIG. 8. If the fourth cardiac rhythm disruptive shock does induce a cardiac rhythm change, when the cardiac controller 206 repeats the process 300, the cardiac controller 206 may apply a sixth cardiac rhythm disruptive shock at a sixth energy level that is higher than the fourth energy level by a predetermined amount (e.g., at 70 J), and so on until the cardiac controller 206 reaches one of the endpoints shown in FIG. 8.

Similarly, if the first cardiac rhythm disruptive shock does induce a cardiac rhythm change in the patient 104, when the cardiac controller 206 repeats the process 300, the cardiac controller 206 may apply a seventh cardiac rhythm disruptive shock at a seventh energy level that is higher than the first energy level by a predetermined amount (e.g., at 100 J). If the seventh cardiac rhythm disruptive shock does not induce a cardiac rhythm change in the patient 104, the cardiac controller 206 may apply an eight cardiac rhythm disruptive shock at an eighth energy level that is lower than the seventh energy level by a predetermined amount (e.g., at 90 J), and so on until the cardiac controller 206 reaches one of the endpoints shown in FIG. 8. If the seventh cardiac rhythm disruptive shock does induce a cardiac rhythm change, when the cardiac controller 206 repeats the process 300, the cardiac controller 206 may apply a ninth cardiac rhythm disruptive shock at a ninth energy level that is higher than the seventh energy level by a predetermined amount (e.g., at 110 J).

In implementations, the cardiac controller 206 may be configured to apply one or more cardiac rhythm restoring shocks to the patient 104 to restore a normal cardiac rhythm in the patient 104 on detecting the cardiac rhythm change in the patient 104 at step 306. As an illustration, if the cardiac rhythm change in the patient 104 is ventricular fibrillation, the cardiac controller 206 may apply one or more defibrillation shocks to the patient 104 to restore the patient's normal cardiac rhythm. If a first defibrillation shock does not restore the patient's normal cardiac rhythm, the cardiac controller 206 may increase the energy level of a subsequent defibrillation shock and continue increasing the energy level of defibrillation shocks until the patient's normal cardiac rhythm is restored. For example, the cardiac controller 206 may increase the energy level of each subsequent defibrillation shock by a predetermined amount (e.g., 30 Joules, 50 Joules, 80 Joules, or other user-configurable energy). As another example, the cardiac controller 206 may increase the energy level of each subsequent defibrillation shock according to a predetermined schedule. For instance, the predetermined schedule may include two or more shocks from the list of 50 Joules, 60 Joules, 80 Joules, 100 Joules, 120 Joules, 150 Joules, 160 Joules, 180 Joules, 200 Joules, 220 Joules, 250 Joules, 260 Joules, 280 Joules, 300 Joules, 320 Joules, 350 Joules, and 360 Joules. In implementations, the cardiac controller 206 may be configured to apply the cardiac rhythm restoring shock after a predetermined delay, such as a time between around 10 ms to 40 ms or a user-configurable time (e.g., received from a clinician via a user terminal 106). The cardiac controller 206 may be further configured to record the energy level of a cardiac rhythm restoring shock (or shocks) that restored the normal cardiac rhythm of the patient 104 in the memory and/or the predetermined delay used for the cardiac rhythm restoring shock or shocks.

The cardiac controller 206 adjusts a defibrillation energy level for one or more future defibrillation shocks to be delivered by the wearable cardiac treatment system (e.g., delivered by the wearable cardiac treatment device 100) at step 312. In implementations, the cardiac controller 206 adjusts the defibrillation energy level for the future defibrillation shocks based on the energy level of the cardiac rhythm disruptive shock that induced the cardiac rhythm change. As an example, the cardiac controller 206 may store an algorithm or a look-up table for determining the defibrillation energy level for future defibrillation shocks. The algorithm or look-up table may correlate the energy level of the cardiac rhythm disruptive shock that induced a cardiac rhythm change in the patient 104 to defibrillation energy levels likely to be successful to treat a cardiac arrhythmia occurring in the patient 104 (e.g., based on studies, likely to be successful 90% of the time, 95% of the time, 98% of the time, etc.). Alternatively, or additionally, the algorithm or look-up table may correlate the energy level of the last cardiac rhythm disruptive shock that did not induce a cardiac rhythm change in the patient 104 to defibrillation energy levels likely to be successful to treat a cardiac arrhythmia occurring in the patient 104. The cardiac controller 206 may thus use the energy level of the cardiac rhythm disruptive shock as an input into the algorithm or look-up table to identify the defibrillation energy level for future defibrillation shocks. Alternatively, in implementations, the remote server 102 may store and use an algorithm or a look-up table to determine the defibrillation energy level for future defibrillation shocks. The remote server 102 may thus receive signals and data from the wearable cardiac treatment device 100, such as ECG signals and/or other biometric or physiological information for the patient 104, determine the defibrillation energy level for future defibrillation shocks from the algorithm or look-up table, and transmit the defibrillation energy level back to the cardiac controller 206.

In implementations, other inputs may be used instead or as well, such as the patient's gender, age, health history (e.g., stage of heart disease), a measured amount of cardiac rhythm change (e.g., the severity of a ventricular fibrillation induced in the patient 104, the number of premature ventricular contractions induced in the patient 104, etc.), a delay used before a cardiac rhythm restoring shock was applied, and/or the like. In implementations, the energy level of a cardiac rhythm restoring shock used to restore the patient's heart to a normal cardiac rhythm may be used as another input into the algorithm or look-up table. As an example, a cardiac rhythm disruptive shock (e.g., a pacing shock delivered asynchronous to the patient's normal cardiac rhythm) may trigger a premature ventricular depolarization in the patient 104. After a premature ventricular depolarization, the subsequent R-R intervals may be irregular as the patient's heart returns to its normal cardiac rhythm. Thus, the wearable cardiac treatment device 100 and/or the remote server 102 may use R-R intervals of the patient's ECG subsequent to the premature ventricular depolarization to calculate one or more heart rate turbulence measures. To illustrate, the wearable cardiac treatment device 100 and/or the remote server 102 may calculate turbulence onset as the difference between the mean of the first two R-R intervals subsequent to a premature ventricular depolarization. As another illustration, the wearable cardiac treatment device 100 and/or the remote server may calculate the turbulence slope as the highest slope of a regression line over any of five consecutive R-R intervals of the twenty R-R intervals subsequent to a premature ventricular depolarization. The cardiac controller 206 and/or remote server 102 may thus use a heart rate turbulence measure (e.g., turbulence onset and/or turbulence slope) as the input to the algorithm or look-up table.

As an illustration of the foregoing, an equation may be produced (e.g., by the remote server 102 or another computer system) using predetermined correlations between various inputs (e.g., energy levels of successful cardiac disruptive shocks, biometric information, physiological information, etc.) and energy levels for defibrillation shocks that successfully restored a normal cardiac rhythm (e.g., with a predetermined amount of certainty, such as 90% certainty) in a patient population. For example, the equation may be a regression function, such as a linear regression function, polynomial regression function, exponential regression function, spline regression function, logarithmic regression function, exponential regression function, power regression function, combinations thereof, and/or the like. Examples of regression functions are provided below, where the X values represent inputs to the function (e.g., energy levels of successful cardiac disruptive shocks, biometric information, physiological information, etc.) and the A, B, C, etc. values represent predetermined constants. For example, the constants may be provided values based on user inputs via user configurable parameters on a user interface.

$$Defibrillation energy level = A * X + B$$

$$Defibrillation energy level = {A}_{1} * {X}_{1} + {A}_{2} * {X}_{2} + B$$

$$Defibrillation energy level = {A}_{1} * {X}_{1} + {A}_{2} * {X}_{2} + … + {A}_{n} * {X}_{n} + B$$

$$Defibrillation energy level = {A}_{1} * {X}_{1} + {A}_{2} * {X}_{2} - B * {X}_{3} + C$$

$$Defibrillation energy level = A * {X}^{B} + C$$

$$Defibrillation energy level = {A}_{1} * {X}_{1}^{{B}_{1}} + {A}_{2} * {X}_{2}^{{B}_{2}} + C$$

$$Defibrillation energy level = {A}_{1} * {X}_{1}^{{B}_{1}} + {A}_{2} * {X}_{2}^{{B}_{2}} + … + {A}_{n} * {X}_{n}^{{B}_{n}} + C$$

$$Defibrillation energy level = A * log ( X ) + B$$

$$Defibrillation energy level = {A}_{1} * log ( {X}_{1} ) + {A}_{2} * log ( {X}_{2} ) + … + {A}_{n} * log ( {X}_{n} ) + B$$

$$Defibrillation energy level = A * {e}^{X} + C$$

$$Defibrillation energy level = A * {e}^{B * X} + C$$

$$Defibrillation energy level = A * {e}^{{B}_{1} * {X}_{1} + {B}_{2} * {X}_{2} + … + {B}_{n} * {X}_{n}} + C$$

As another example, the cardiac controller 206 may repeat steps 304-310 a predetermined number of times, as discussed above. The cardiac controller 206 may then use the cardiac rhythm disruptive shocks that successfully induced a cardiac rhythm change (and/or the last cardiac rhythm disruptive shocks that did not induce a cardiac rhythm change) and a dose-response curve to identify an energy level percentile for the patient. For example, the cardiac controller 206 may determine the 50th percentile energy level shown to be effective in the baseline therapy energy session, which may approximate or be used to approximate a certain level of the DFT for the patient (e.g., the DFT90). As such, the dose-response curve may be another example of an algorithm or look-up table used to determine the defibrillation energy level for future defibrillation shocks for the patient 104.

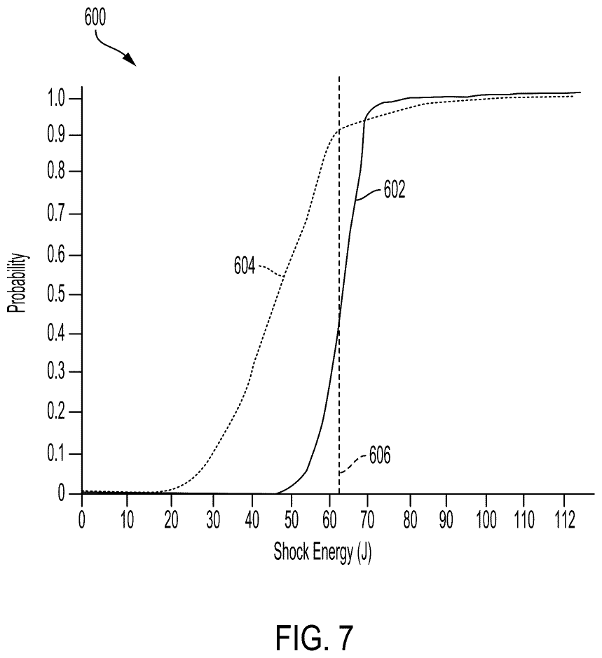

As an illustration, FIG. 7 shows an example dose-response curve 600 for a hypothetical patient. The dose-response curve 600 includes a curve 602 representing for cardiac rhythm disruptive shocks (e.g., defibrillation-like shocks intended to cause an episode of VF) plotted against the probability of the cardiac rhythm disruptive shocks at the different energy levels (here, measured in Joules) inducing or not inducing a cardiac rhythm disruption (e.g., an episode of VF). In implementations, the curve 602 represents the probability of not inducing VF at or above the different energy levels presented on the x-axis. In the example of FIG. 7, the probability is the probability that the cardiac rhythm disruptive shock does not induce a cardiac rhythm disruption. As shown in the example of FIG. 7, the curve 602 for the cardiac rhythm disruptive shocks has a low probability (e.g., at or near 0) of not inducing a cardiac rhythm disruption at lower energy levels (e.g., below around 45 Joules), has an increasing probability of not inducing a cardiac rhythm disruption between around 45 Joules and 75 Joules, and has a high probability (e.g., at or near 1) of not inducing a cardiac rhythm disruption at higher energy levels (e.g., above around 75 Joules). However, the curve 602 for the cardiac rhythm disruptive shocks may have different thresholds and slopes for different patients. For instance, the thresholds at which the curve changes slope may be different for different patients (e.g., depending on cardiac structure, body impedance, etc.). As another example, the slope of the increasing probability portion of the curve 602 may be different for different patients.

The example dose-response curve 600 also includes a “success” curve 604 for defibrillation shock energies plotted against the probability of the defibrillation shock energies at the different energy levels successfully restoring the example patient to a normal cardiac rhythm. In the example of FIG. 7, the curve 604 for the defibrillation shocks has a similar shape to the curve 602, though with different thresholds and a different slope. As shown in FIG. 7, the curve 604 for the defibrillation shocks has a low probability (e.g., at or near 0) of successfully restoring the patient to a normal cardiac rhythm at lower energy levels (e.g., below around 20 Joules), has an increasing probability of restoring the patient to a normal cardiac rhythm between around 20 Joules and 90 Joules, and has a high probability of restoring the patient to a normal cardiac rhythm at higher energy levels (e.g., above around 90 Joules). As with the curve 602 for cardiac rhythm disruptive shocks (e.g., for non-VF-inducing shocks), the curve 604 for defibrillation shocks may have different thresholds and slopes for different patients.

Additionally, the dose-response curve 600 includes a line 606 marking the fiftieth percentile for the cardiac rhythm disruptive shocks curve 602. As shown by the line 606, the shock energy level at the 50th percentile (e.g., around 63 Joules) has been shown to correspond to the 90th percentile for the defibrillation shocks curve 604. Although the curves 602 and 604 may look slightly different for different patients, the same principle may hold true. As such, by determining the energy level at or above which a cardiac rhythm disruptive shock does not induce a cardiac rhythm change about half of the time (e.g., the energy level at which a fibrillation shock induces an episode of ventricular fibrillation about half of the time) for a particular patient, the cardiac controller 206 and/or the remote server 102 can determine the energy level at or above which a defibrillation shock is expected to be successful 90% of the time in restoring the patient to a normal cardiac rhythm about ninety percent of the time. The benefit of constructing the curve 602 to determine this energy level is the convenience of constructing the curve 602 compared to the difficulty of measuring the defibrillation energies for restoring the patient to a normal cardiac rhythm required to construct the curve 604 (e.g., which would require inducing a ventricular fibrillation state more times compared to the baselining process described above).

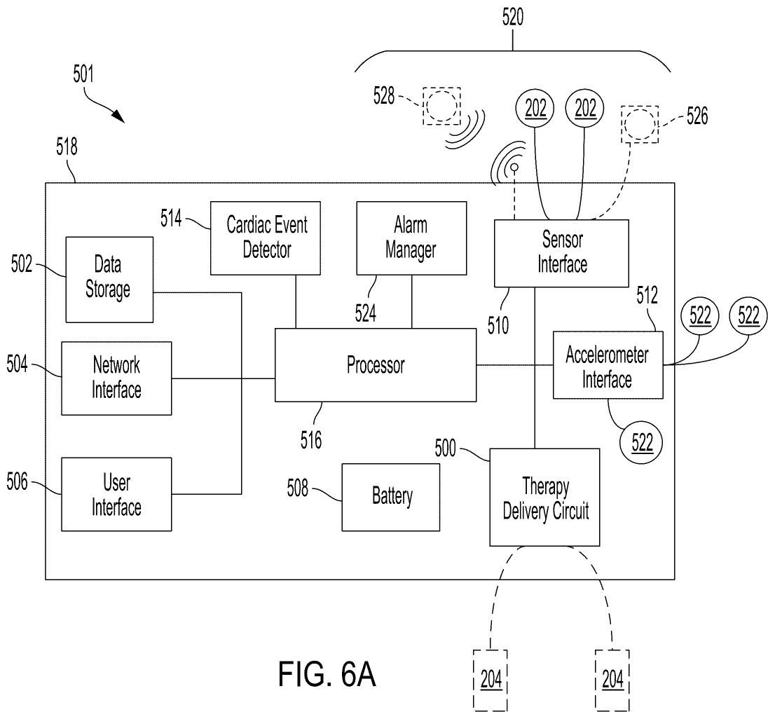

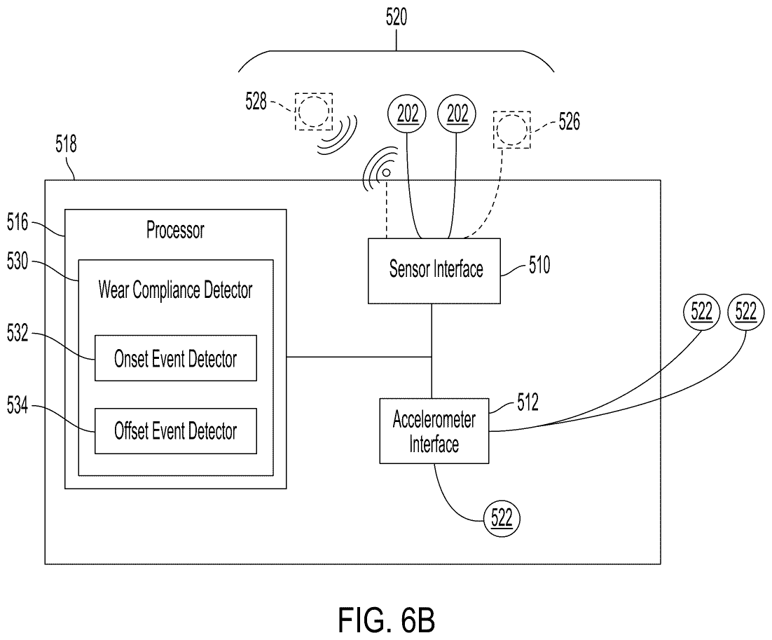

To illustrate, a wearable cardiac treatment device 100 may apply a series of decreasing cardiac rhythm disruptive shocks until a cardiac rhythm disruptive shock induces a cardiac rhythm change in the patient 104, as described above with reference to steps 304-310. The wearable cardiac treatment device 100 may repeat the process of applying a series of decreasing cardiac rhythm disruptive shocks until a cardiac rhythm disruptive shock induces a cardiac rhythm change in the patient 104 at least one additional time. The wearable cardiac treatment device 100 and/or the remote server 102 may then take use the percentage of time that a given energy level did not induce a cardiac rhythm change in the patient 104 to construct a dose-response curve for the patient 104, similar to the dose-response curve 600 shown in FIG. 6. The wearable cardiac treatment device 100 and/or remote server may then determine the 50th percentile of the dose-response curve. Because the 50th percentile of the dose-response curve for cardiac rhythm disruptive shocks approximates the 90th percentile of the dose-response curve for the defibrillation shocks for the patient 104, the wearable cardiac treatment device 100 may set the energy level of the 50th percentile as the defibrillation energy level for future defibrillation shocks.

As an example, of the foregoing, the wearable cardiac treatment device 100 may perform a baselining session with a patient 104, where the results of the baselining session are shown below in Table 1. In this example baselining session, the wearable cardiac treatment device 100 repeated the process of applying cardiac rhythm disruptive shocks six times, starting at 100 J and decreasing each successive cardiac rhythm disruptive shock by 10 J if the patient 104 did not show a cardiac rhythm change (e.g., an episode of VF). As shown in Table 1, the results of the baselining session are as follows: in the first test, the patient 104 entered VF when the first shock at 100 J was delivered. In the second test, the patient 104 received three shocks and entered VF when the third shock at 80 J was delivered. In the third and fourth tests, the patient 104 received four shocks and entered VF when the fourth shock at 70 J was delivered. In the fifth test, the patient 104 received five shocks and entered VF when the fifth shock at 60 J was delivered. In the sixth test, the patient 104 received two shocks and entered VF when the second shock at 90 J was delivered. Table 1 also includes the probability of each energy level not inducing an episode of VF in the patient 104. These probabilities are calculated as the percentage of time the given energy level did not induce an episode of VF across the six tests. If a given energy level was not applied to the patient 104 during a given test because the patient 104 had already entered VF due to a higher energy level being applied, that given energy level is considered as having induced VF in the patient 104 if applied for that test.

TABLE 1
Example Results for a Baselining Session

| Energy | Test | Test | Test | Test | Test | Test | Probability |
|---|---|---|---|---|---|---|---|
| level (J) | 1 | 2 | 3 | 4 | 5 | 6 | of no VF |
| 100 | 1 | 0 | 0 | 0 | 0 | 0 | 83% |
| 90 | | 0 | 0 | 0 | 0 | 1 | 66% |
| 80 | | 1 | 0 | 0 | 0 | | 50% |
| 70 | | | 1 | 1 | 0 | | 17% |
| 60 | | | | | 1 | | 0% |
| 50 | | | | | | | 0% |
| 40 | | | | | | | 0% |

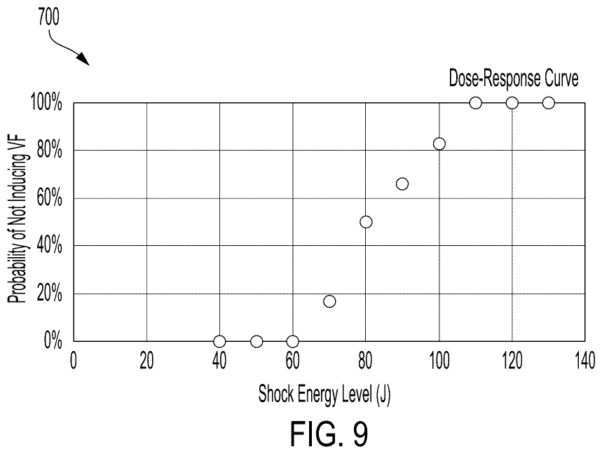

FIG. 9 illustrates an example dose-response curve 700 constructed for the patient 104 from the example baselining session data shown in Table 1 (with additional data points added for 110 J and 120 J, which are assumed to be at a 100% probability of not causing VF). In implementations, the wearable cardiac treatment device 100 and/or the remote server 102 may determine the 50th percentile of the dose-response curve 700 through extrapolation, such as by constructing a trendline for the dose-response curve 700 and determining what shock energy level corresponds to 50% on the trendline. For example, the cardiac controller 206 and/or the remote server 102 may use a curve fitting function (e.g., curve fitting to a logistic function, curve fitting to a spline, curve fitting to a linear regression) to create a full dose-response curve from the graph shown in FIG. 9. In implementations, the wearable cardiac treatment device 100 and/or the remote server 102 may determine the 50th percentile of the dose-response curve by finding the approximate 50% probability in the data shown in Table 1. In this example, 80 J corresponds to 50% probability. However, if the data did not include an exact 50% probability, the wearable cardiac treatment device 100 and/or the remote server 102 may take an average or a weighted average between the energy levels above and below the 50% probability mark as approximating the 50th percentile for the patient 104. For instance, if the results of a baselining session showed that 70 J corresponded to a 66% probability and that 60 J corresponded to a 33% probability, the wearable cardiac treatment device 100 and/or the remote server 102 may determine that 65 J approximates the 50% probability mark.

Other methods may be used to determine or approximate the 50th percentile of the dose-response curve, however. For example, in implementations, the wearable cardiac treatment device 100 may repeat the process of applying cardiac rhythm disruptive shocks according to the schedule shown in FIG. 8 until the wearable cardiac treatment device 100 reaches one of the endpoints of the schedule. The wearable cardiac treatment device 100 (and/or the remote server 102) may then determine that the endpoint approximates the 50th percentile of the dose-response curve for the patient 104. In implementations, the wearable cardiac treatment device 100 and/or the remote server 102 may determine a different predetermined percentile of the dose-response curve to use in adjusting the future defibrillation shock energy.

In implementations, the wearable cardiac treatment device 100 may set an iteration of the 50th percentile of the dose-response curve as the defibrillation energy level for future defibrillation shocks. The iteration may be, for instance, the 50th percentile added to a predetermined amount (e.g., 3 Joules, 5 Joules, 10 Joules, 20 Joules, 30 Joules, 40 Joules, 50 Joules, 60 Joules, 70 Joules, 80 Joules, 90 Joules, 100 Joules, etc., or other user configurable energy) and/or the 50th percentile modified by a multiplier (e.g., 1.2 times, 1.5 times, 1.8 times, 2 times, 3 times, etc. the average or median). In implementations, the 50th percentile may instead be used as an input into a regression function, as described above.

In implementations, the cardiac controller 206 may use energy levels of cardiac rhythm restoring shocks applied to the patient 104 to restore the patient's normal heart rhythm after an induced cardiac rhythm change to determine the energy level for future defibrillation shocks. As an illustration, the cardiac controller 206 may apply a first cardiac rhythm restoring shock at a first restoring shock energy level, detect that the patient 104 has not been restored to a normal cardiac rhythm, and apply a second cardiac rhythm restoring shock at a second restoring shock energy level. For instance, the first restoring shock energy level may be around 20 to 90 Joules, and the second restoring shock energy level may be around 20 to 90 Joules. As another illustration, the first restoring shock energy level may be around 20 to 90 Joules, and the second restoring shock energy level may be around 70 to 90 Joules. As another illustration, the first restoring shock energy level may be around 20 to 90 Joules (e.g., 50 Joules), and the second restoring shock energy level may be around 100 to 200 Joules (e.g., 150 Joules). The cardiac controller 206 may also apply more than two cardiac rhythm restoring shocks to the patient 104 at increasingly higher energy levels if the first two cardiac rhythm restoring shocks did not restore the patient 104 to a normal cardiac rhythm.

The cardiac controller 206 may record, in the memory of the wearable cardiac treatment system, the restoring shock energy level of the cardiac rhythm restoring shock that restored the patient to the normal cardiac rhythm state. The cardiac controller 206 may further adjust the defibrillation energy level for one or more future defibrillation shocks to be delivered by the wearable cardiac treatment system based on the restoring shock energy level of the cardiac rhythm restoring shock that restored the patient 104 to the normal cardiac rhythm state, such as by using the restoring shock energy level as an input to the algorithm, look-up table, or patient-specific dose-response curve correlating cardiac rhythm disruptive shock energy levels to successful defibrillation shock energy levels. In implementations, the cardiac controller 206 may also use the restoring shock energy levels of any cardiac rhythm disruptive shocks that did not restore the patient 104 to the normal cardiac rhythm as, for example, an input to an algorithm, look-up table, or patient-specific dose-response curve.

As noted above, in implementations, the cardiac controller 206 may be configured to repeat steps 304-310 of process 300, thus, repeating applying the at least one or the series of cardiac rhythm disruptive shocks, detecting when a cardiac rhythm change has occurred in the patient 104, and recording the energy level of the cardiac rhythm disruptive shock that induced the cardiac rhythm change. The cardiac controller 206 and/or remote server 102 may be configured to perform a statistical analysis on the energy levels of the cardiac rhythm disruptive shocks that induced the cardiac rhythm changes and adjust the defibrillation energy level for the one or more future defibrillation shocks based on the statistical analysis. For example, the cardiac controller 206 and/or remote server 102 may find an average, a median, a mode, a highest energy level, etc. of the energy levels of the cardiac disruptive shocks that induced the cardiac rhythm changes in the patient 104. Performing this statistical analysis may further include eliminating any outlier cardiac disruptive shocks that induced a cardiac rhythm change and finding, for example, the average, median, highest energy level, etc. of the energy levels of the remaining cardiac disruptive shocks that induced the cardiac rhythm changes in the patient 104. The cardiac controller 206 and/or remote server 102 may then use the result of the statistical analysis as the input or as an input to an algorithm, look-up table, or patient-specific dose-response curve correlating cardiac rhythm disruptive shock energy levels to successful defibrillation shock energy levels. Alternatively, in implementations, performing a statistical analysis may include constructing a dose-response curve similar to the curves 600 and 700 shown in FIGS. 7 and 9, respectively, and determining the 50th percentile of the dose-response curve, as discussed above.

As another example, the cardiac controller 206 and/or remote server 102 may construct another type of dose-response curve for the patient 104 (e.g., different from the dose-response curves shown in FIGS. 7 and 9) using any of the potential inputs discussed above (e.g., energy levels of cardiac rhythm disruptive shocks that did induce a cardiac rhythm change, measures of heart rate turbulence, successful and/or unsuccessful cardiac rhythm restoring shocks, outputs of statistical analyses of these values, etc.). Constructing the dose-response curve may include, for example, the cardiac controller 206 and/or remote server 102 using a curve fitting function (e.g., curve fitting to a logistic function, curve fitting to a spline, curve fitting to a linear regression) to create a dose-response curve from a graph of these potential inputs. The dose-response curve may be used to determine the energy level for future defibrillation shocks or to determine a further input to an algorithm or look-up table.

In implementations, the wearable cardiac treatment device 100 may take one or more impedance measurements from the patient 104 at the time that the wearable cardiac treatment device 100 performs the baseline therapy session with the patient 104. For example, the wearable cardiac treatment device 100 may transmit a signal with a known current and waveform shape (e.g., a square wave) into the patient 104, such as via the ECG electrodes 202 or via the therapy electrodes 204. The wearable cardiac treatment device 100 may then detect a voltage from the patient 104 (e.g., via the ECG electrodes 202 and/or via the therapy electrodes 204) and convert the detected voltage into the patient's impedance using Ohm's law. As another example, the wearable cardiac treatment device 100 may detect a voltage from the patient 104 after delivering one or more cardiac rhythm disruptive shocks and/or cardiac rhythm restoring shocks to the patient 104 and convert the detected voltage into the patient's impedance using Ohm's law. Therefore, when the wearable cardiac treatment device 100 adjusts the defibrillation energy level for future defibrillation shocks at step 312, the wearable cardiac treatment device 100 may also determine the voltage and/or current of the defibrillation shocks to be delivered to the patient 104 using the patient's known impedance.

In implementations, the cardiac controller 206 and/or remote server 102 may also determine a delay to be used between the detection of a treatable cardiac arrhythmia and the application of the defibrillation shock. For example, the cardiac controller 206 may determine the delay using the same algorithm, look-up table, or patient-specific dose-response curve used to identify the defibrillation energy level for future shocks (e.g., the delay may be another output of the algorithm, look-up table, or patient-specific dose-response curve). As another example, the cardiac controller 206 may use delays for cardiac rhythm restoring shocks used to restore the patient to a normal cardiac sinus rhythm to identify a delay time period that was more or most effective for the patient during the baseline therapy energy session.

In various implementations discussed above, the cardiac controller 206 may be the component determining the defibrillation energy level for future defibrillation shocks. In some implementations, the remote server 102 may instead determine the defibrillation energy level for future defibrillation shocks. For example, the cardiac controller 206 may transmit the energy level of a cardiac rhythm disruptive shock that induced a cardiac rhythm change to the remote server 102 to be stored in a memory of the remote server 102. The remote server 102 may then use the energy level of the cardiac rhythm disruptive shock to determine a defibrillation energy level for future defibrillation shocks (e.g., using an algorithm, look-up table, or patient-specific dose-response curve as discussed above). The remote server 102 may transmit the determined defibrillation energy level to the wearable cardiac treatment device 100, which stores the determined defibrillation energy level (e.g., in the data storage 502 of the cardiac controller 206).

To illustrate a commercial application of the foregoing, the patient 104 may be fitted with a wearable cardiac treatment device 100 and sedated (e.g., as shown in FIG. 1). A clinician or technician (e.g., the technician 108 shown in FIG. 1) may initiate a baseline therapy energy session (e.g., by pressing a button on the cardiac controller 206, by navigating to a menu selection on a display of the cardiac controller 206). The cardiac controller 206 may then navigate the wearable cardiac treatment device 100 through the baseline therapy energy session by applying a series of cardiac rhythm disruptive shocks to the patient 104, as described above, and a series of cardiac rhythm restoring shocks to the patient 104 (if needed). Once the baseline therapy energy session has been completed, the wearable cardiac treatment device 100 may output a recommended defibrillation energy level for the patient 104 (e.g., with the recommended defibrillation energy level being the output of step 312 of FIG. 4).

In implementations, the wearable cardiac treatment device 100 may automatically set the recommended defibrillation energy level as the future defibrillation energy level for the patient. In implementations, the wearable cardiac treatment device 100 may provide the recommended defibrillation energy level to a clinician (e.g., by displaying the recommended defibrillation energy level on the cardiac controller 206, by transmitting the recommended defibrillation energy level to a clinician-authorized user terminal 106). The clinician may then accept the recommended defibrillation energy level or adjust the defibrillation energy level, after which the wearable cardiac treatment device 100 receives the adjusted defibrillation energy level and sets the adjusted defibrillation energy level as the future defibrillation energy level for the patient 104.

Example pseudocode for the wearable cardiac treatment device 100 performing a baseline therapy session is provided below:

```
  set fibrillation_number to 1;
  while (fibrillation_number < 6) {
    set fibrillation_energy_level to 100 J;
    set fibrillation_event to FALSE;
    while (fibrillation_event = FALSE) {
      execute fibrillation shock at fibrillation_energy_level;
      retrieve ECG segment corresponding to time of
fibrillation shock;
      perform a fibrillation analysis on ECG segment;
        if the patient had a ventricular fibrillation event
{
          execute defibrillation shock;
          record the fibrillation_energy_level;
          set fibrillation_event to TRUE
        }
        else set fibrillation_energy_level to
fibrillation_energy_level − 10 J;
    }
    set fibrillation_number to fibrillation_number + 1;
  }
  find dose-response probabilities for fibrillation_energy_level
amounts;
  determine 50th percentile from dose-response probabilities;
  set a recommended defibrillation energy level to double the 50th
percentile;
  output the recommended defibrillation energy level;
```

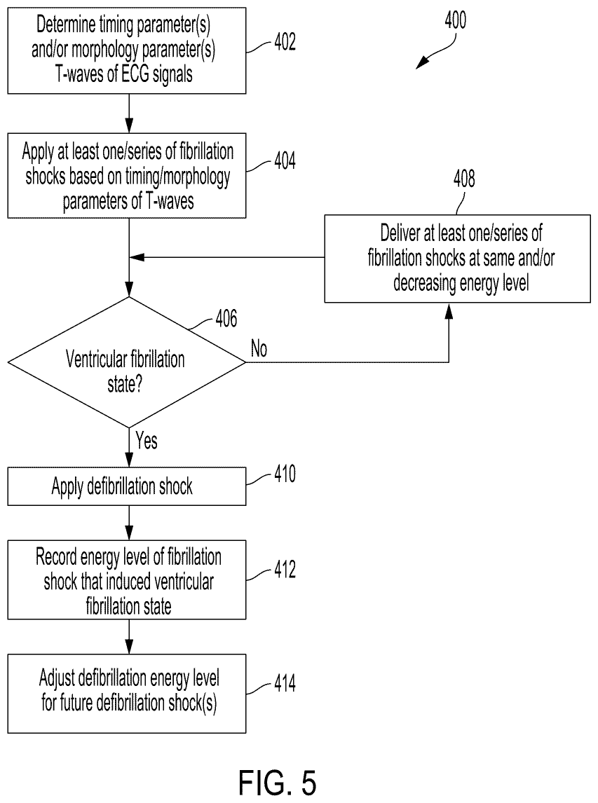

FIG. 5 illustrates a sample process flow for performing a baseline therapy energy session where the desired cardiac rhythm change is ventricular fibrillation. Similar to the sample process 300 of FIG. 4, the sample process 400 can be implemented by the cardiac controller 206. The cardiac controller 206 determines one or more timing parameters and/or one or more morphology parameters of T-waves in the patient 104 based on the ECG signals at step 402. In embodiments, the cardiac controller 206 may implement step 402 similarly to step 302 of process 300, with the one or more timing parameters and/or one or more morphology parameters identified corresponding to the T-waves in the patient 104.

The cardiac controller 206 applies at least one or a series of fibrillation shocks at predetermined one or more times based on the one or more timing parameters and/or one or more morphology parameters of the T-waves in the patient 104 at step 404. In embodiments, the cardiac controller 206 may implement step 404 similarly to step 304 of process 300, with the at least one or the series of fibrillation shocks being applied to correspond with T-waves of the patient 104 to attempt to induce a ventricular fibrillation state in the patient 104. The cardiac controller 206 determines whether the patient 104 has entered a ventricular fibrillation state at step 406. For example, the cardiac controller 206 may apply one or more ventricular fibrillation templates to the patient's ECG signals to identify whether the ECG signals show that the patient 104 has entered into ventricular fibrillation. If the cardiac controller 206 determines that the patient has not entered a ventricular fibrillation state, the cardiac controller 206 delivers at least one or a series of fibrillation shocks at same and/or decreasing energy levels until the patient 104 enters the ventricular fibrillation state (or the baseline therapy energy process is stopped or aborted) at step 408. The cardiac controller 206 may implement step 408 similarly to step 308 of process 300.

When the cardiac controller 206 determines that the patient 104 has entered the ventricular fibrillation state, the cardiac controller 206 applies a defibrillation shock to the patient 104 via the therapy electrodes 204 to treat the ventricular fibrillation state. In implementations, the cardiac controller 206 may apply more than one defibrillation shock to the patient 104 at increasing energy levels until the patient 104 is restored to a normal cardiac sinus rhythm, as discussed above with respect to FIG. 4. In implementations, the cardiac controller 206 may apply the defibrillation shock to the patient 104 after a predetermined delay, as also discussed above with respect to FIG. 4, at step 410.

The cardiac controller 206 records an energy level of a fibrillation shock that induced the ventricular fibrillation state as the baseline energy information in a memory of the wearable cardiac treatment system at step 412. The cardiac controller 206 may implement step 412 similarly to step 310 of process 300, as discussed above. The cardiac controller 206 also adjusts a defibrillation energy level for one or more future defibrillation shocks to be delivered by the wearable cardiac treatment system (e.g., by the wearable cardiac treatment device 100) based on the energy level of the fibrillation shock that induced the ventricular fibrillation state. The cardiac controller may implement step 414 similarly to step 312 of process 300, as discussed above. For example, the cardiac controller 206 and/or the remote server 102 may construct a dose-response curve similar to the dose-response curve 600 shown in FIG. 7 to determine the defibrillation energy level for one or more future defibrillation shocks.

Returning to the wearable cardiac treatment device 100, FIG. 6A illustrates a sample component-level view of a medical device controller 501 included in a wearable cardiac treatment device 100. The medical device controller 501 is an example of the cardiac controller 206 shown in FIG. 3 and described above. As shown in FIG. 6A, the medical device controller 501 can include a housing 518 configured to house a therapy delivery circuit 500 configured to provide one or more therapeutic shocks to the patient 104 via the therapy electrodes 204, a data storage 502, a network interface 504, a user interface 506, at least one battery 508 (e.g., within a battery chamber configured for such purpose), a sensor interface 510 (e.g., to interface with the ECG electrodes 202 and other physiological sensors or detectors such as vibrational sensors, lung fluid sensors, infrared and near-infrared-based pulse oxygen sensors, and blood pressure sensors, among others), a cardiac event detector 514, an alarm manager 524, and at least one processor 516. As described above, in some implementations, the wearable cardiac treatment device 100 that includes like components as those described above but does not include the therapy delivery circuit 500 and the therapy electrodes 204 (shown in dotted lines). That is, in some implementations, the wearable cardiac treatment device 100 can include the ECG monitoring components and not provide therapy to the patient.

The therapy delivery circuit 500 can be coupled to the therapy electrodes 204 configured to provide therapy to the patient 104. For example, the therapy delivery circuit 500 can include, or be operably connected to, circuitry components that are configured to generate and provide an electrical therapeutic shock. The circuitry components can include, for example, resistors, capacitors, relays and/or switches, electrical bridges such as an h-bridge (e.g., including a plurality of insulated gate bipolar transistors or IGBTs), voltage and/or current measuring components, and other similar circuitry components arranged and connected such that the circuitry components work in concert with the therapy delivery circuit 500 and under the control of one or more processors (e.g., processor 516) to provide, for example, one or more pacing, defibrillation, or cardioversion therapeutic pulses. In implementations, pacing pulses can be used to treat cardiac arrhythmias such as bradycardia (e.g., less than 30 beats per minute) and tachycardia (e.g., more than 150 beats per minute) using, for example, fixed rate pacing, demand pacing, anti-tachycardia pacing, and the like. Defibrillation or cardioversion pulses can be used to treat ventricular tachycardia and/or ventricular fibrillation. In implementations, the therapy delivery circuit 500 is also configured to deliver the cardiac rhythm disruptive shocks (e.g., defibrillation-like shocks, pacing pulses, etc.) discussed above.

The capacitors can include a parallel-connected capacitor bank consisting of a plurality of capacitors (e.g., two, three, four, or more capacitors). In some examples, the capacitors can include a single film or electrolytic capacitor as a series connected device including a bank of the same capacitors. These capacitors can be switched into a series connection during discharge for a defibrillation pulse. For example, four capacitors of approximately 140 uF or larger, or four capacitors of approximately 650 uF can be used. The capacitors can have a 1600 VDC or higher rating for a single capacitor, or a surge rating between approximately 350 to 500 VDC for paralleled capacitors and can be charged in approximately 15 to 30 seconds from a battery pack.

For example, each defibrillation pulse can deliver between 60 to 180 J of energy. In some implementations, the defibrillating pulse can be a biphasic truncated exponential waveform, whereby the signal can switch between a positive and a negative portion (e.g., charge directions). This type of waveform can be effective at defibrillating patients at lower energy levels when compared to other types of defibrillation pulses (e.g., such as monophasic pulses). For example, an amplitude and a width of the two phases of the energy waveform can be automatically adjusted to deliver a precise energy amount (e.g., 150 J) regardless of the patient's body impedance. The therapy delivery circuit 500 can be configured to perform the switching and pulse delivery operations, e.g., under control of the processor 516. As the energy is delivered to the patient 104, the amount of energy being delivered can be tracked. For example, the amount of energy can be kept to a predetermined constant value even as the pulse waveform is dynamically controlled based on factors, such as the patient's body impedance, while the pulse is being delivered.

In certain examples, the therapy delivery circuit 500 can be configured to deliver a set of cardioversion pulses to correct, for example, an improperly beating heart. When compared to defibrillation as described above, cardioversion typically includes a less powerful shock that is delivered at a certain frequency to mimic a heart's normal rhythm.

The data storage 502 can include one or more of non-transitory computer-readable media, such as flash memory, solid state memory, magnetic memory, optical memory, cache memory, combinations thereof, and others. The data storage 502 can be configured to store executable instructions and data used for operation of the medical device controller 501. In some implementations, the data storage 502 can include sequences of executable instructions that, when executed, are configured to cause the processor 516 to perform one or more functions. For example, the data storage 502 can be configured to store information such as ECG data as received from, for instance, the sensor interface 510.

In some examples, the network interface 504 can facilitate the communication of information between the cardiac controller 206 and one or more devices or entities over a communications network. For example, the network interface 504 can be configured to communicate with the remote server 102 or other similar computing device. The network interface 504 can include communications circuitry for transmitting data in accordance with a Bluetooth® wireless standard for exchanging such data over short distances to an intermediary device(s) (e.g., a base station, “hotspot” device, smartphone, tablet, portable computing device, and/or other device in proximity with the wearable cardiac treatment device 100). The intermediary device(s) may in turn communicate the data to the remote server 102 over a broadband cellular network communications link. The communications link may implement broadband cellular technology (e.g., 2.5G, 2.75G, 3G, 4G, 5G cellular standards) and/or Long-Term Evolution (LTE) technology or GSM/EDGE and UMTS/HSPA technologies for high-speed wireless communication. In some implementations, the intermediary device(s) may communicate with the remote server 102 over a Wi-Fi communications link based on the IEEE 802.11 standard. In some implementations, the network interface 504 may be configured to instead communicate directly with the remote server 102 without the use of intermediary device(s). In such implementations, the network interface 504 may use any of the communications links and/or protocols provided above.

In some implementations, the user interface 506 may include one or more physical interface devices, such as input devices, output devices, and combination input/output devices, and a software stack configured to drive operation of the devices. These user interface elements may render visual, audio, and/or tactile content. Thus, the user interface 506 may receive inputs and/or provide outputs, thereby enabling a user to interact with the cardiac controller 206.

The cardiac controller 206 can also include at least one battery 508 configured to provide power to one or more components integrated in the cardiac controller 206. The battery 508 can include a rechargeable multi-cell battery pack. In one example implementation, the battery 508 can include three or more cells (e.g., 2200 mA lithium ion cells) that provide electrical power to the other device components within the cardiac controller 206. For example, the battery 508 can provide its power output in a range of between 20 mA to 1000 mA (e.g., 40 mA) output and can support 24 hours, 48 hours, 72 hours, or more, of runtime between charges. In certain implementations, the battery capacity, runtime, and type (e.g., lithium ion, nickel-cadmium, or nickel-metal hydride) can be changed to best fit the specific application of the cardiac controller 206.

The sensor interface 510 can include physiological signal circuitry that is coupled to one or more externally worn sensors configured to monitor one or more physiological parameters of the patient and output one or more physiological signals. As shown, the sensors may be coupled to the medical device controller 501 via a wired or wireless connection. The sensors can include one or more ECG electrodes 202 (e.g., ECG electrodes) configured to output at least one ECG signal. In some implementations, the sensors can include conventional ECG sensing electrodes and/or digital sensing electrodes. The sensors can also include one or more non-ECG physiological sensors 520 such as one or more vibration sensors 526, tissue fluid monitors 528 (e.g., based on ultra-wide band RF devices), one or more motion sensors (e.g., accelerometers, gyroscopes, and/or magnetometers), a temperature sensor, a pressure sensor, a P-wave sensor (e.g., a sensor configured to monitor and isolate P-waves within an ECG waveform), an oxygen saturation sensor (e.g., implemented through photoplethysmography, such as through light sources and light sensors configured to transmit light into the patient's body and receive transmitted and/or reflected light containing information about the patient's oxygen saturation), and so on.

The one or more vibration sensors 526 can be configured to detect cardiac or pulmonary vibration information. For example, the vibration sensors 526 can detect a patient's heart valve vibration information. For example, the vibration sensors 526 can be configured to detect cardio-vibrational signal values including any one or all of S1, S2, S3, and S4. From these cardio-vibrational signal values or heart vibration values, certain heart vibration metrics may be calculated, including any one or more of electromechanical activation time (EMAT), average EMAT, percentage of EMAT (% EMAT), systolic dysfunction index (SDI), and left ventricular systolic time (LVST). The vibration sensors 526 can also be configured to detect heart wall motion, for instance, by placement of the sensor in the region of the apical beat. The vibration sensors 526 can include a vibrational sensor configured to detect vibrations from a patient's cardiac and pulmonary system and provide an output signal responsive to the detected vibrations of a targeted organ, for example, being able to detect vibrations generated in the trachea or lungs due to the flow of air during breathing. In certain implementations, additional physiological information can be determined from pulmonary-vibrational signals such as, for example, lung vibration characteristics based on sounds produced within the lungs (e.g., stridor, crackle, etc.). The vibration sensors 526 can also include a multi-channel accelerometer, for example, a three-channel accelerometer configured to sense movement in each of three orthogonal axes such that patient movement/body position can be detected and correlated to detected cardio-vibrations information. The vibration sensors 526 can transmit information descriptive of the cardio-vibrations information to the sensor interface 510 for subsequent analysis.

The tissue fluid monitors 528 can use RF based techniques to assess fluid levels and accumulation in a patient's body tissue. For example, the tissue fluid monitors 528 can be configured to measure fluid content in the lungs, typically for diagnosis and follow-up of pulmonary edema or lung congestion in heart failure patients. The tissue fluid monitors 528 can include one or more antennas configured to direct RF waves through a patient's tissue and measure output RF signals in response to the waves that have passed through the tissue. In certain implementations, the output RF signals include parameters indicative of a fluid level in the patient's tissue. The tissue fluid monitors 528 can transmit information descriptive of the tissue fluid levels to the sensor interface 510 for subsequent analysis.

The controller 501 can further include a motion detector interface operably coupled to one or more motion detectors configured to generate motion data, for example, indicative of physical activity performed by the patient 104. Examples of a motion detector may include a 1-axis channel accelerometer, 2-axis channel accelerometer, 3-axis channel accelerometer, multi-axis channel accelerometer, gyroscope, magnetometer, ballistocardiograph, and the like. As an illustration, the motion data may include accelerometer counts indicative of physical activity, accelerometer counts indicative of respiration rate, and posture information for the patient 104. For instance, in some implementations, the controller 501 can include an accelerometer interface 512 operably coupled to one or more accelerometers 522, as shown in FIG. 6A. Alternatively, in some implementations, the accelerometer interface 512 may be incorporated into other components of the controller 501. As an example, the accelerometer interface 512 may be part of the sensor interface 510, and the one or more accelerometers 522 may be part of the non-ECG physiological sensors 520.

The accelerometer interface 512 is configured to receive one or more outputs from the accelerometers. The accelerometer interface 512 can be further configured to condition the output signals by, for example, converting analog accelerometer signals to digital signals (if using an analog accelerometer), filtering the output signals, combining the output signals into a combined directional signal (e.g., combining each x-axis signal into a composite x-axis signal, combining each y-axis signal into a composite y-axis signal, and combining each z-axis signal into a composite z-axis signal). In some examples, the accelerometer interface 512 can be configured to filter the signals using a high-pass or band-pass filter to isolate the acceleration of the patient due to movement from the component of the acceleration due to gravity.

Additionally, the accelerometer interface 512 can configure the output for further processing. For example, the accelerometer interface 512 can be configured to arrange the output of an individual accelerometer 522 as a vector expressing the acceleration components of the x-axis, the y-axis, and the z-axis as received from each accelerometer. The accelerometer interface 512 can be operably coupled to the processor 516 and configured to transfer the output signals from the accelerometers 522 to the processor for further processing and analysis.

The one or more accelerometers 522 can be integrated into one or more components of the wearable cardiac treatment device 100. In some implementations, one or more motion detectors 522 may be located in or near the ECG electrodes 202. In some implementations, the one or more motion detectors 522 may be located elsewhere on the wearable cardiac treatment device 100. For example, a motion detector 522 can be integrated into the controller 501. In some examples, a motion detector 522 can be integrated into one or more of a therapy electrode 204, an ECG electrode 202, the connection pod 208, and/or into other components of the wearable cardiac treatment device 100. In some examples, a motion detector 522 can be integrated into an adhesive ECG sensing and/or therapy electrode patch.

As described above, the sensor interface 510 and the accelerometer interface 512 can be coupled to any one or combination of sensing electrodes/other sensors to receive patient data indicative of patient parameters. Once data from the sensors has been received by the sensor interface 510 and/or the accelerometer interface 512, the data can be directed by the processor 516 to an appropriate component within the medical device controller 501. For example, ECG signals collected by the ECG electrodes 202 may be transmitted to the sensor interface 510, and the sensor interface 510 can transmit the ECG signals to the processor 516, which, in turn, relays the data to the cardiac event detector 514. The sensor data can also be stored in the data storage 502 and/or transmitted to the remote server 102 via the network interface 504. For instance, the processor 516 may transfer the ECG signals from the ECG electrodes 202 and the motion data from the one or more accelerometers 522 to the remote server 102.

In implementations, the cardiac event detector 514 can be configured to monitor the patient's ECG signal for an occurrence of a cardiac event such as an arrhythmia or other similar cardiac event. The cardiac event detector can be configured to operate in concert with the processor 516 to execute one or more methods that process received ECG signals from, for example, the ECG electrodes 202 and determine the likelihood that a patient is experiencing a cardiac event, such as a treatable arrhythmia. The cardiac event detector 514 can be implemented using hardware or a combination of hardware and software. For instance, in some examples, cardiac event detector 514 can be implemented as a software component that is stored within the data storage 502 and executed by the processor 516. In this example, the instructions included in the cardiac event detector 514 can cause the processor 516 to perform one or more methods for analyzing a received ECG signal to determine whether an adverse cardiac event is occurring, such as a treatable arrhythmia. In other examples, the cardiac event detector 514 can be an application-specific integrated circuit (ASIC) that is coupled to the processor 516 and configured to monitor ECG signals for adverse cardiac event occurrences. Thus, examples of the cardiac event detector 514 are not limited to a particular hardware or software implementation.

In response to the cardiac event detector 514 determining that the patient 104 is experiencing a treatable arrhythmia, the processor 516 is configured to deliver a cardioversion/defibrillation shock to the patient 104 via the therapy electrodes 204. In some implementations, the alarm manager 524 can be configured to manage alarm profiles and notify one or more intended recipients of events, where an alarm profile includes a given event and the intended recipients who may have in interest in the given event. These intended recipients can include external entities, such as users (e.g., patients, physicians and other caregivers, a patient's loved one, monitoring personnel), as well as computer systems (e.g., monitoring systems or emergency response systems, which may be included in the remote server 102 or may be implemented as one or more separate systems). For example, when the processor 516 determines using data from the ECG electrodes 202 that the patient is experiencing a treatable arrhythmia, the alarm manager 524 may issue an alarm via the user interface 506 that the patient is about to experience a defibrillating shock. The alarm may include auditory, tactile, and/or other types of alerts. In some implementations, the alerts may increase in intensity over time, such as increasing in pitch, increasing in volume, increasing in frequency, switching from a tactile alert to an auditory alert, and so on. Additionally, in some implementations, the alerts may inform the patient that the patient can abort the delivery of the defibrillating shock by interacting with the user interface 506. For instance, the patient may be able to press a user response button or user response buttons on the user interface 506, after which the alarm manager 524 will cease issuing an alert and the cardiac controller 206 will no longer prepare to deliver the defibrillating shock.

In implementations, the cardiac event detector 514 is configured to detect when the patient 104 is experiencing a cardiac rhythm change (e.g., an episode of VF, an episode of VT, a premature ventricular contraction) in response to a cardiac rhythm disruptive shock (e.g., coordinated by the therapy delivery circuit 500) delivered during a baselining session, as discussed above. Depending on the type of cardiac rhythm change, the processor 516 is configured to deliver a cardioversion/defibrillation shock to the patient 104 via the therapy electrodes 204, as discussed above, to restore the patient's normal cardiac rhythm. For example, if the cardiac rhythm change is VF, the processor 516 is configured to deliver a cardioversion/defibrillation shock to the patient 104. The processor 516 is also configured to record, in the data storage 502, data related to the cardiac rhythm change and the cardiac rhythm disruptive shock, as further discussed above (e.g., the energy level of the cardiac rhythm disruptive shock that induced the cardiac rhythm change).

The alarm manager 524 can be implemented using hardware or a combination of hardware and software. For instance, in some examples, the alarm manager 524 can be implemented as a software component that is stored within the data storage 502 and executed by the processor 516. In this example, the instructions included in the alarm manager 524 can cause the processor 516 to configure alarm profiles and notify intended recipients using the alarm profiles. In other examples, the alarm manager 524 can be an application-specific integrated circuit (ASIC) that is coupled to the processor 516 and configured to manage alarm profiles and notify intended recipients using alarms specified within the alarm profiles. Thus, examples of the alarm manager 524 are not limited to a particular hardware or software implementation.

In some implementations, the processor 516 includes one or more processors (or one or more processor cores) that each are configured to perform a series of instructions that result in the manipulation of data and/or the control of the operation of the other components of the medical device controller 501. In some implementations, when executing a specific process (e.g., cardiac monitoring), the processor 516 can be configured to make specific logic-based determinations based on input data received. The processor 516 may be further configured to provide one or more outputs that can be used to control or otherwise inform subsequent processing to be carried out by the processor 516 and/or other processors or circuitry with which the processor 516 is communicably coupled. Thus, the processor 516 reacts to a specific input stimulus in a specific way and generates a corresponding output based on that input stimulus. In some example cases, the processor 516 can proceed through a sequence of logical transitions in which various internal register states and/or other bit cell states internal or external to the processor 516 may be set to logic high or logic low.

As referred to herein, the processor 516 can be configured to execute a function where software is stored in a data store (e.g., the data storage 502) coupled to the processor 516, the software being configured to cause the processor 516 to proceed through a sequence of various logic decisions that result in the function being executed. The various components that are described herein as being executable by the processor 516 can be implemented in various forms of specialized hardware, software, or a combination thereof. For example, the processor 516 can be a digital signal processor (DSP) such as a 24-bit DSP processor. As another example, the processor 516 can be a multi-core processor, e.g., having two or more processing cores. As another example, the processor 516 can be an Advanced RISC Machine (ARM) processor, such as a 32-bit ARM processor. The processor 516 can execute an embedded operating system and further execute services provided by the operating system, where these services can be used for file system manipulation, display and audio generation, basic networking, firewalling, data encryption, communications, and/or the like.

As noted above, a wearable cardiac treatment device, such as the wearable cardiac treatment device 100, can be designed to include a digital front-end where analog signals sensed by skin-contacting electrode surfaces of a set of digital sensing electrodes are converted to digital signals for processing. Typical ambulatory medical devices with analog front-end configurations use circuitry to accommodate a signal from a high source impedance from the sensing electrode (e.g., having an internal impedance range from approximately 100 Kiloohms to one or more Megaohms). This high source impedance signal is processed and transmitted to a monitoring device such as processor 516 of the controller 501 as described above for further processing. In certain implementations, the monitoring device, or another similar processor such as a microprocessor or another dedicated processor operably coupled to the sensing electrodes, can be configured to receive a common noise signal from each of the sensing electrodes, sum the common noise signals, invert the summed common noise signals and feed the inverted signal back into the patient as a driven ground using, for example, a driven right leg circuit to cancel out common mode signals.

The wearable cardiac treatment device 100 is configured for long-term and/or extended use or wear by, or attachment or connection to, a patient. For example, devices as described herein may be capable of being continuously used or continuously worn by, or attached or connected to a patient, without substantial interruption (e.g., up to 24 hours or beyond, such as for weeks, months, or even years). In some implementations, such devices may be removed for a period of time before use, wear, attachment, or connection to the patient is resumed. As an illustration, devices may be removed to change batteries, carry out technical service, update the device software or firmware, and/or to take a shower or engage in other activities, without departing from the scope of the examples described herein. Such substantially or nearly continuous use or wear as described herein may nonetheless be considered continuous use or wear. Additionally, the wearable cardiac treatment device 100 may be configured to transmit signals and data to the remote server 102 continuously or substantially continuously.

As described herein, and noted above, implementations of the present disclosure include monitoring medical device wear compliance for the patient 104. More specifically, the wear compliance information includes an accurate overview of what portion or percentage of a certain time period the patient has worn the wearable cardiac treatment device 100 and how this compares to the expected wear for the patient 104 as prescribed, for example, by their clinician or other healthcare provider when being prescribed the wearable cardiac treatment device 100. FIG. 6B illustrates an example reduced component-level view of the medical device controller 501 that includes the processor 516 that is configured to monitor wear compliance information for the patient 104 as described herein. For example, the processor 516 can include wear time circuitry, such as a wear compliance detector 530 as shown in FIG. 6B. The wear compliance detector 530 may be integrated into the processor 516 as illustrated in FIG. 6B, or the wear compliance detector 530 may be integrated as a separate processing component operably coupled to the processor 516. The wear compliance detector 530 can be implemented as a dedicated microprocessor and associated circuitry disposed on a printed circuit board (PCB) along with other components as described herein. The wear compliance detector 530, when implemented in a dedicated microprocessor or integrated into the processor 516, can be based on a series of processor-readable instructions configured to be executed by the dedicated microprocessor or processor 516. For example, the instructions can be implemented in a programming language such as C, C++, assembly language, machine code, HDL, or VHDL. In examples, the dedicated microprocessor can be an Intel-based microprocessor such as an X86 microprocessor or a Motorola 68020 microprocessor, each of which can use a different set of binary codes and/or instructions for similar functions. In implementations, the dedicated microprocessor or processor 516 can be configured to implement wear onset event detection and wear offset event detection as set forth in FIG. 6B above.

As further shown in FIG. 6B, the wear compliance detector 530 can include an onset event detector 532 and an offset event detector 534. As described above, the wear compliance detector 530 can be a dedicated microprocessor and associated circuitry disposed on a PCB along with other components as described herein. In implementations, a first microprocessor can be implemented as the onset event detector 532, and a second microprocessor can be implemented as the offset event detector 534. In some implementations, both the onset event detector 532 and offset event detector 534 can be implemented in the same microprocessor as described above. The onset event detector 532 and/or offset event detector 534, when implemented in a dedicated microprocessor or integrated into the processor 516, can be based on a series of processor-readable instructions configured to be executed by the dedicated microprocessor or processor 516.

As noted above, when a patient puts on the wearable cardiac treatment device 100, a wear onset event can be determined based upon analysis of signals received from one or more of the sensors described herein. For example, based upon monitoring of signals output by the ECG electrodes 202 as well as signals output by the accelerometers 522, the onset event detector 532 can determine an onset event indicative of the patient 104 putting on or otherwise wearing the wearable cardiac treatment device 100. Similarly, the offset event detector 534 can determine an offset event indicative of the patient 104 turning off, removing, or otherwise stopping the wearable cardiac treatment device 100 from monitoring. Based upon the measured onset and offset events, the wear compliance detector 530 and/or the processor 516 can determine wear compliance information (e.g., wear time) for the patient 104.

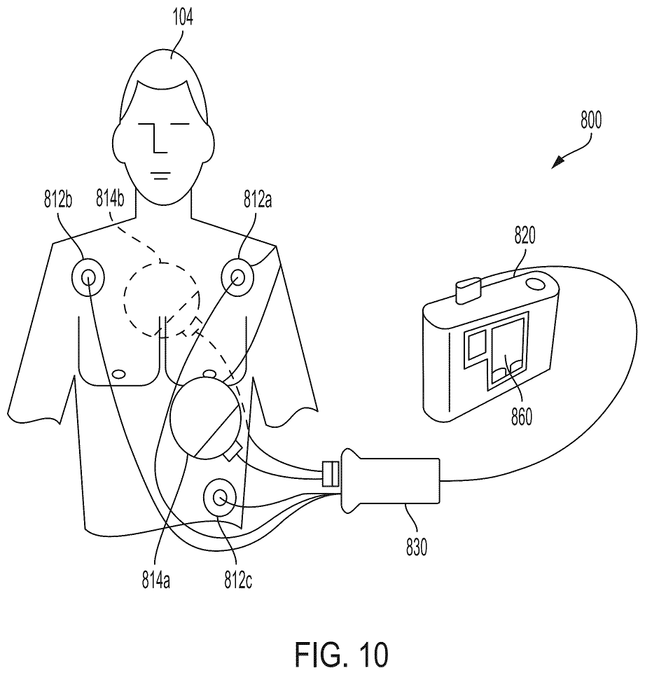

FIG. 10 illustrates another example of a wearable cardiac treatment device 100. More specifically, FIG. 10 shows a hospital wearable defibrillator 800 that is external, ambulatory, and wearable by the patient 104. Hospital wearable defibrillator 800 can be configured in some implementations to provide pacing therapy, e.g., to treat bradycardia, tachycardia, and asystole conditions. The hospital wearable defibrillator 800 can include one or more ECG sensing electrodes 812a, 812b, 812c (e.g., collectively ECG sensing electrodes 812), one or more therapy electrodes 814a and 814b (e.g., collectively therapy electrodes 814), a medical device controller 820, and a connection pod 830. For example, each of these components can be structured and function as similar components of the embodiments of the wearable cardiac treatment device 100 discussed above with reference to FIGS. 3 and 6A-6B. In implementations, the electrodes 812 and 814 can include disposable adhesive electrodes. For example, the electrodes can include sensing and therapy components disposed on separate sensing and therapy electrode adhesive patches. In some implementations, both sensing and therapy components can be integrated and disposed on a same electrode adhesive patch that is then attached to the patient. For example, the front adhesively attachable therapy electrode 814a attaches to the front of the patient's torso to deliver pacing or defibrillating therapy. Similarly, the back adhesively attachable therapy electrode 814b attaches to the back of the patient's torso. In an example scenario, at least three ECG adhesively attachable sensing electrodes 812 can be attached to at least above the patient's chest near the right arm (e.g., electrode 812b), above the patient's chest near the left arm (e.g., electrode 812a), and towards the bottom of the patient's chest (e.g., electrode 812c) in a manner prescribed by a trained professional.

A patient being monitored by a hospital wearable defibrillator and/or pacing device may be confined to a hospital bed or room for a significant amount of time (e.g., 75% or more of the patient's stay in the hospital). As a result, a user interface 860 can be configured to interact with a user other than the patient (e.g., a technician, a clinician or other caregiver) for device-related functions such as initial device baselining (e.g., including performing a baselining therapy session), setting and adjusting patient parameters, and changing the device batteries.

In some implementations, an example of a therapeutic medical device that includes a digital front-end in accordance with the systems and methods described herein can include a short-term defibrillator and/or pacing device. For example, such a short-term device can be prescribed by a physician for patients presenting with syncope. A wearable defibrillator can be configured to monitor patients presenting with syncope by, e.g., analyzing the patient's physiological and cardiac activity for aberrant patterns that can indicate abnormal physiological function. For example, such aberrant patterns can occur prior to, during, or after the onset of syncope. In such an example implementation of the short-term wearable defibrillator, the electrode assembly can be adhesively attached to the patient's skin and otherwise have a similar configuration or functionality as the wearable cardiac treatment device 100 described above in connection with FIGS. 3 and 6A-6B.

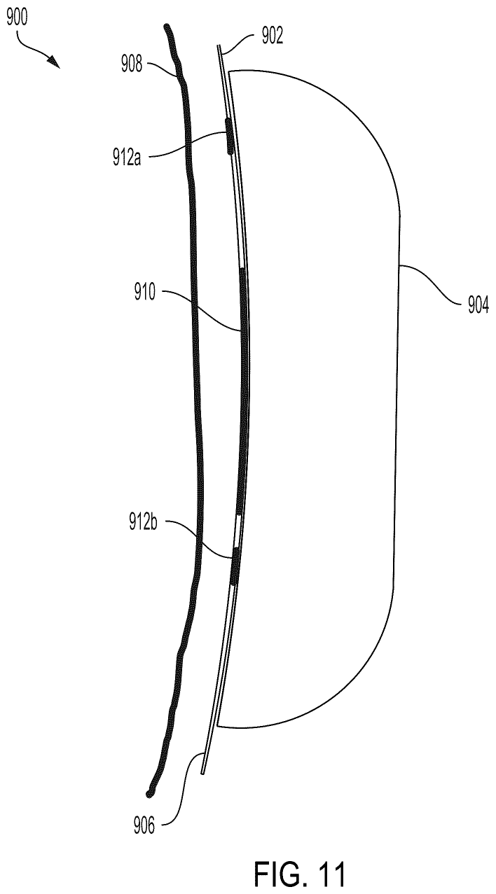

FIG. 11 illustrates another example of a wearable cardiac treatment device 100. As shown in FIG. 11, the wearable cardiac treatment device 100 may be or include an adhesive assembly 900. The adhesive assembly 900 includes a contoured pad 902 and a housing 904 configured to form a watertight seal with the contoured pad 902. In implementations, the housing 904 is configured to house electronic components of the adhesive assembly, such as electronic components similar to components described above with respect to FIGS. 6A and 6B. The adhesive assembly 900 includes a conductive adhesive layer 906 configured to adhere the adhesive assembly 900 to a skin surface 908 of the patient 104. The adhesive layer 906 may include, for example, a water-vapor permeable conductive adhesive material, such as a material selected from the group consisting of an electro-spun polyurethane adhesive, a polymerized microemulsion pressure sensitive adhesive, an organic conductive polymer, an organic semi-conductive conductive polymer, an organic conductive compound and a semi-conductive conductive compound, and combinations thereof.

The adhesive assembly 900 also includes at least one therapy electrode 910 integrated with the contoured pad 902. In implementations, the adhesive assembly 900 may include a therapy electrode 910 that forms a vector with another therapy electrode disposed on another adhesive assembly 900 adhered to the patient's body and/or with a separate therapy electrode adhered to the patient's body (e.g., similar to therapy electrodes 814a and 814b of FIG. 10). The adhesive assembly 900 may also include one or more ECG sensing electrodes 912 integrated with the contoured pad 902 (e.g., ECG sensing electrodes 912a and 912b). In implementations, the adhesive assembly 900 may alternatively or additionally be in electronic communication with a separate ECG sensing electrode, such as an adhesive sensing electrode adhered to the patient's body. In examples, as shown in FIG. 11, the therapy electrode(s) 910 and ECG sensing electrode(s) 912 may be formed within the contoured pad 902 such that a skin-contacting surface of each component is coplanar with or protrudes from the patient-contacting face of the contoured pad 902. Examples of a wearable cardiac treatment device 100 including an adhesive assembly 900 are described in U.S. patent application Ser. No. 16/585,344, entitled “Adhesively Coupled Wearable Medical Device,” filed on Sep. 27, 2019, which is hereby incorporated by reference in its entirety.

Although the subject matter contained herein has been described in detail for the purpose of illustration, such detail is solely for that purpose and that the present disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

Other examples are within the scope and spirit of the description and claims. Additionally, certain functions described above can be implemented using software, hardware, firmware, hardwiring, or combinations of any of these. Features implementing functions can also be physically located at various positions, including being distributed such that portions of functions are implemented at different physical locations.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. Those skilled in the art will readily appreciate that all parameters, dimensions, materials, and configurations described herein are meant to be an example and that the actual parameters, dimensions, materials, and/or configurations will depend upon the specific application or applications for which the inventive teachings is/are used.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

1-101. (canceled)

102. A wearable cardiac treatment system configured to treat arrhythmias occurring in an ambulatory patient, comprising:
a garment configured to be worn about a torso of the ambulatory patient;
a plurality of ECG electrodes configured to be disposed on the garment and further configured to sense ECG signals indicative of cardiac activity in the patient;
a plurality of therapy electrodes configured to be disposed on the garment;
a memory configured to store baseline therapy energy information; and
a cardiac controller comprising one or more processors in communication with the memory, the plurality of ECG electrodes and the plurality of therapy electrodes, the one or more processors configured to
determine one or more timing parameters and/or one or more morphology parameters of the ECG signals of the patient;
apply, via the plurality of therapy electrodes, at least one or a series of cardiac rhythm disruptive shocks at predetermined one or more times based on the one or more timing parameters and/or one or more morphology parameters of the ECG signals of the patient, wherein the at least one or the series of the cardiac rhythm disruptive shocks are delivered at same and/or decreasing energy levels until a cardiac rhythm change is induced in the patient,
detect the cardiac rhythm change in the patient,
record, in the memory, an energy level of a cardiac rhythm disruptive shock that induced the cardiac rhythm change as the baseline therapy energy information, and
adjust a defibrillation energy level for one or more future defibrillation shocks to be delivered by the wearable cardiac treatment system based on the energy level of the cardiac rhythm disruptive shock that induced the cardiac rhythm change.

103. The wearable cardiac treatment system of claim 102, wherein the one or more processors are configured to apply, via the plurality of therapy electrodes, the at least one or the series of cardiac rhythm disruptive shocks by
applying a first cardiac rhythm disruptive shock at a first energy level; and
detecting that no cardiac rhythm change has occurred in the patient.

104. The wearable cardiac treatment system of claim 103, wherein the one or more processors are further configured to apply, via the plurality therapy electrodes, the at least one or the series of cardiac rhythm disruptive shocks by applying a second cardiac rhythm disruptive shock at a second energy level lower than the first energy level.

105. The wearable cardiac treatment system of claim 104, wherein the first energy level comprises an energy level between around 20 to 90 Joules and the second energy level comprises an energy level between around 20 to 90 Joules.

106. The wearable cardiac treatment system of claim 105, wherein the first energy level comprises an energy level between around 70 to 90 Joules and the second energy level comprises an energy level between around 30 to 50 Joules.

107. The wearable cardiac treatment system of claim 102, wherein the one or more processors are further configured to apply, via the plurality of therapy electrodes, a cardiac rhythm restoring shock to the patient to restore a normal cardiac rhythm on detecting the cardiac rhythm change in the patient.

108. The wearable cardiac treatment system of claim 107, wherein the one or more processors are configured to apply, via the one or more therapy electrodes, the cardiac rhythm restoring shock to restore the normal cardiac rhythm by
applying a first cardiac rhythm restoring shock at a first restoring shock energy level;
detecting that the patient has not been restored to the normal cardiac rhythm; and
applying a second cardiac rhythm restoring shock at a second restoring shock energy level higher than the first restoring shock energy level.

109. The wearable cardiac treatment system of claim 108, wherein the one or more processors are further configured to record, in the memory, a restoring shock energy level of the cardiac rhythm restoring shock that restored the patient to the normal cardiac rhythm.

110. The wearable cardiac treatment system of claim 109, wherein the one or more processors are configured to further adjust the defibrillation energy level for one or more future defibrillation shocks to be delivered by the wearable cardiac treatment system based on the restoring shock energy level of the cardiac rhythm restoring shock that restored the patient to the normal cardiac rhythm.

111. The wearable cardiac treatment system of claim 102, wherein the one or more processors are configured to apply, via the plurality of therapy electrodes, the at least one or the series of cardiac rhythm disruptive shocks by applying a first cardiac rhythm disruptive shock at a first energy level, the first cardiac rhythm disruptive shock inducing the cardiac rhythm change in the patient.

112. The wearable cardiac treatment system of claim 108, wherein the one or more processors are further configured to apply, via the plurality of therapy electrodes, a second cardiac rhythm disruptive shock at a second energy level higher than the first energy level.

113. The wearable cardiac treatment system of claim 102, wherein the one or more processors are further configured to repeat applying the at least one or the series of cardiac rhythm disruptive shocks, detecting the cardiac rhythm change in the patient, and recording the energy level of the cardiac rhythm disruptive shock that induced the cardiac rhythm change.

114. The wearable cardiac treatment system of claim 113, wherein the one or more processors are further configured to construct a dose-response curve for the patient based on the recorded energy levels of the cardiac rhythm disruptive shocks that induced the cardiac rhythm changes.

115. The wearable cardiac treatment system of claim 114, wherein the one or more processors are further configured to determine a predetermined percentile of the dose-response curve.

116. The wearable cardiac treatment system of claim 115, wherein the predetermined percentile comprises a 50th percentile.

117. The wearable cardiac treatment system of claim 115, wherein the one or more processors are configured to adjust the defibrillation energy level for the one or more future defibrillation shocks to be delivered by the wearable cardiac treatment system using an energy level corresponding to the predetermined percentile of the dose-response curve.

118. The wearable cardiac treatment system of claim 102, wherein the cardiac rhythm change in the patient comprises at least one of ventricular fibrillation or a premature ventricular contraction.

119. The wearable cardiac treatment system of claim 102, wherein the one or more timing parameters and/or one or more morphology parameters of the ECG signals comprise one or more timings and/or one or more morphologies corresponding to one or more T-waves in the patient.

120. The wearable cardiac treatment system of claim 102, wherein the cardiac controller further comprises the memory configured to store the baseline therapy energy information.

121. The wearable cardiac treatment system of claim 102, further comprising a remote server comprising the memory configured to store the baseline therapy energy information.
